(19) United States
(12) Patent Application Publication
(10) Pub. No.: US 2024/0287623 A1
(43) Pub. Date: Aug. 29, 2024

(54) SIGNAL BOOST ASSAY PERFORMED IN DROPLETS

(71) Applicant: VedaBio, Inc., San Diego, CA (US)

(72) Inventors: Don Masquelier, San Diego, CA (US); Phillip Belgrader, San Diego, CA (US); Anurup Ganguli, San Diego, CA (US)

(21) Appl. No.: 18/586,420

(22) Filed: Feb. 23, 2024

Related U.S. Application Data
(60) Provisional application No. 63/449,006, filed on Feb. 28, 2023.

(51) Int. Cl. C12Q 1/689; C12Q 1/6823; C12Q 1/6827

(52) U.S. Cl. CPC C12Q 1/689; C12Q 1/6823; C12Q 1/6827; C12Q 2600/156

(57) ABSTRACT

The present disclosure relates to multiplex assay methods and systems used to detect several to many to a massively multiplexed number of target nucleic acids of interest in a sample without amplification of the target nucleic acids of interest. The method employs microfluidic droplet systems where each droplet is a “mini-reactor.” In some embodiments, a “bulk format” configuration is used, in other embodiments, a “sequential format” configuration is used.

### RELATED APPLICATIONS

This application claims priority to U.S. Ser. No. 63/449,006, filed 28 Feb. 2023, which is incorporated by reference in its entirety.

### INCORPORATION BY REFERENCE OF SEQUENCE LISTING

Submitted herewith is an electronically filed sequence listing via EFS-Web a Sequence Listing XML, entitled “VB017US_seq_list_20240223”, created 23 Feb. 2024, which is 12,288 bytes in size. The sequence listing is part of the specification of this specification and is incorporated by reference in its entirety.

### FIELD OF THE INVENTION

The present disclosure relates to multiplexed assay methods used to detect and identify several to many to a massively multiplexed number of target nucleic acids of interest in a sample without amplification of the target nucleic acids of interest.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Rapid and accurate identification of, e.g., infectious agents, microbe contamination, variant nucleic acid sequences that indicate the presence of diseases such as cancer, and/or contamination by heterologous sources is important in order to select correct treatment; identify tainted food, pharmaceuticals, cosmetics and other commercial goods; and to monitor the environment including identification of biothreats. Classic PCR and nucleic acid-guided nuclease or CRISPR (clustered regularly interspaced short palindromic repeats) detection methods rely on pre-amplification of target nucleic acids of interest to enhance detection sensitivity. However, amplification increases time to detection and may cause changes to the relative proportion of nucleic acids in samples that, in turn, lead to artifacts or inaccurate results. Improved technologies that allow very rapid and accurate detection of nucleic acids are therefore needed for timely diagnosis and treatment of disease, to identify toxins in consumables and the environment, as well as in other applications.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure provides multiplexed microfluidic cascade assay methods to detect target nucleic acids of interest in a sample without amplification of the target nucleic acids of interest. The “nucleic acid-guided nuclease cascade assays” or “signal boost cascade assays” or “signal boost assays” or “cascade assays” described herein comprise two different ribonucleoprotein (RNP) complexes and either blocked nucleic acid molecules, blocked primer molecules, or blocked guide nucleic acids, all of which allow for massive multiplexing. This signal boost cascade assay is combined with a microfluidic droplet system that generates droplet “mini-reactors” that allow one to test for tens, hundreds and even thousands of different target nucleic acids of interest in a single assay. The blocked nucleic acid molecules, blocked primer molecules, or blocked guide nucleic acids in the signal boost cascade assay keep second ribonucleoprotein complexes (RNP2s) “locked” unless and until a target nucleic acid of interest activates the first ribonucleoprotein complexes (RNP1s). A particularly advantageous feature of the cascade assay is that, with the exception of the gRNAs in RNP1, the cascade assay components may be the same in each assay no matter what target nucleic acids of interest are being detected; moreover, the gRNAs in the RNP1 are easily reprogrammed using traditional guide design methods.

Using a microfluidic droplet system, the results of the signal boost cascade assay can be determined readily, even when assaying for a massive number of target nucleic acids of interest. The exemplary microfluidic droplet systems and method described herein utilize either a “bulk format” or a “sequential format”, as detailed infra, some embodiments allow for identification of target nucleic acids of interest directly from the microfluidic droplet device.

Thus, in a first exemplary embodiment, there is provided a method for identifying one or more target nucleic acids of in a sample comprising the steps of: designing first guide nucleic acids (gRNA1s) complementary to the target nucleic acids of interest; forming first ribonucleoprotein complexes (RNP1s) comprising a first nucleic acid-guided nuclease and the gRNA1s; wherein the first nucleic acid-guided nuclease exhibits both cis- and trans-cleavage activity and wherein the RNP1s are formed in partitions where different partitions comprise different gRNA1 sequences; providing a reaction mixture comprising: the sample; second ribonucleoprotein complexes (RNP2s) comprising a second nucleic acid-guided nuclease and a second gRNA that is not complementary to the target nucleic acids of interest; wherein the second nucleic acid-guided nuclease exhibits both cis- and trans-cleavage activity; a plurality of blocked nucleic acid molecules comprising a sequence complementary to the second gRNA, wherein the blocked nucleic acid molecules comprise: a first region recognized by the second gRNA of the RNP2 complex and one or more second regions not complementary to the first region forming at least one loop; and a plurality of reporter moieties comprising a detectable signal wherein the detectable signal is activated by the trans-cleavage activity of the RNP1s and/or RNP2s; providing a microfluidic droplet system comprising a main flow channel, an RNP1 introduction channel and at least one oil flow channel; introducing a first aqueous fluid through the main flow channel, wherein the first aqueous fluid comprises the reaction mixture; introducing a second aqueous fluid through the RNP1 introduction channel into the first aqueous fluid in the main flow channel, wherein the second aqueous fluid comprises RNP1 complexes with a first gRNA; following introduction of the second aqueous fluid into the main flow channel, introducing a carrier fluid through one or more carrier fluid introduction channels into the main flow channel thereby forming aqueous droplets comprising reaction mixture and RNP1s in the carrier fluid; providing conditions for the one or more target nucleic acids of interest in the sample, if present, to bind to the RNP1s; and detecting the detectable signal, if present, in the aqueous droplets.

In some aspects of this first exemplary embodiment, the method further comprises, after the step of flowing the second aqueous fluid through the RNP1 introduction channel, the step of flowing a first slug fluid through the RNP1 introduction channel and into the first aqueous fluid in the main flow channel, wherein the first slug fluid does not comprise RNP1s. Also in some aspects of this first exemplary embodiment, the first slug fluid may be aqueous or may be carrier fluid and may have a detectable property. Further, in some aspects of this first embodiment, after flowing the first slug fluid through the RNP1 introduction channel, the method further comprises the step of flowing a third aqueous fluid through the RNP1 flow channel and into the first aqueous fluid in the main flow channel, wherein the third aqueous fluid comprises RNP1 complexes with a second gRNA, and, after the step of flowing the third aqueous fluid through the RNP1 introduction channel, the step of flowing a second slug fluid through the RNP1 introduction channel and into the first aqueous fluid in the main flow channel, wherein the second slug fluid does not comprise RNP1s.

In some aspects of this embodiment, the forming step is performed where the partitions are reservoirs coupled by valves to the RNP1 introduction channel.

A second exemplary embodiment provides a method for identifying one or more target nucleic acids in a sample comprising the steps of: designing a plurality of first guide nucleic acids (gRNA1s) complementary to the target nucleic acids of interest; forming first ribonucleoprotein complexes (RNP1s) comprising a first nucleic acid-guided nuclease and the gRNA1s, wherein the first nucleic acid-guided nuclease exhibits both cis- and trans-cleavage activity and wherein the RNP1s are formed in partitions where different partitions comprise different gRNA1 sequences; coupling the first ribonucleoprotein complexes (RNP1s) to carrier beads in the different partitions, wherein the carrier beads in different partitions comprise different nucleic acid barcodes thereby forming RNP1/carrier beads; combining the RNP1/carrier beads from the partitions forming an RNP1/carrier bead library; providing a reaction mixture comprising: the sample; second ribonucleoprotein complexes (RNP2s) comprising a second nucleic acid-guided nuclease and a second gRNA that is not complementary to the target nucleic acids of interest; wherein the second nucleic acid-guided nuclease exhibits both cis- and trans-cleavage activity; a plurality of blocked nucleic acid molecules comprising a sequence complementary to the second gRNA, wherein the blocked nucleic acid molecules comprise: a first region recognized by the second gRNA of the RNP2 complex and one or more second regions not complementary to the first region forming at least one loop; and a plurality of reporter moieties comprising a detectable signal wherein the detectable signal is activated by the trans-cleavage activity of the RNP1s and/or RNP2s; providing a microfluidic droplet system comprising a main flow channel, an RNP1 introduction channel and at least one carrier fluid flow channel; introducing a first aqueous fluid through the main flow channel, wherein the first aqueous fluid comprises the reaction mixture; introducing a second aqueous fluid through the RNP1 introduction channel into the first aqueous fluid in the main flow channel, wherein the second aqueous fluid comprises the RNP1/carrier bead library; following introduction of the second aqueous fluid into the main flow channel, introducing a carrier fluid through one or more carrier fluid introduction channels into the main flow channel thereby forming aqueous droplets in the carrier fluid, wherein the aqueous droplets comprise reaction mixture and a Poisson or super Poisson distribution of RNP1/carrier beads; providing conditions for the target nucleic acids of interest in the sample, if present, to bind to the RNP1s; and detecting the detectable signal, if present, in the aqueous droplets.

A third exemplary embodiment provides a method for identifying one or more target nucleic acids of in a sample comprising the steps of: designing a plurality of first guide nucleic acids (gRNA1s) complementary to the target nucleic acids of interest; forming first ribonucleoprotein complexes (RNP1s) comprising a first nucleic acid-guided nuclease and the gRNA1s; wherein the first nucleic acid-guided nuclease exhibits both cis- and trans-cleavage activity and wherein the RNP1s are formed in partitions where different partitions comprise different gRNA1 sequences; forming second ribonucleoprotein complexes (RNP2s) comprising a second nucleic acid-guided nuclease and a second gRNA that is not complementary to the target nucleic acids of interest; wherein the second nucleic acid-guided nuclease exhibits both cis- and trans-cleavage activity; coupling the first ribonucleoprotein complexes (RNP1s) and second ribonucleoprotein complexes (RNP2s) to carrier beads in the different partitions, wherein the carrier beads in different partitions comprise different nucleic acid barcodes thereby forming RNP1/RNP2/carrier beads; combining the RNP1/RNP2/carrier beads from the partitions forming an RNP1/RNP2/carrier bead library; providing a reaction mixture comprising: the sample; a plurality of blocked nucleic acid molecules comprising a sequence complementary to the second gRNA, wherein the blocked nucleic acid molecules comprise: a first region recognized by the second gRNA of the RNP2 complex and one or more second regions not complementary to the first region forming at least one loop; and a plurality of reporter moieties comprising a detectable signal wherein the detectable signal is activated by the trans-cleavage activity of the RNP1s and/or RNP2s; providing a microfluidic droplet system comprising a main flow channel, an RNP1 introduction channel and a carrier fluid flow channel; introducing a first aqueous fluid through the main flow channel, wherein the first aqueous fluid comprises the reaction mixture; introducing a second aqueous fluid through the RNP1 introduction channel into the first aqueous fluid in the main flow channel, wherein the second aqueous fluid comprises the RNP1/RNP2/carrier bead library; following introduction of the second aqueous fluid into the main flow channel, introducing a carrier fluid through one or more carrier fluid introduction channels into the main flow channel thereby forming aqueous droplets in the carrier fluid, wherein the aqueous droplets comprise reaction mixture and a Poisson or super Poisson distribution of RNP1/RNP2/carrier beads; providing conditions for the target nucleic acids of interest in the sample, if present, to bind to the RNP1s; and detecting fluorescence in the aqueous droplets.

In yet a fourth exemplary embodiment, there is provided a method for identifying one or more target nucleic acids of in a sample comprising the steps of: designing a plurality of first guide nucleic acids (gRNA1s) complementary to the target nucleic acids of interest; forming first ribonucleoprotein complexes (RNP1s) comprising a first nucleic acid-guided nuclease and the gRNA1s; wherein the first nucleic acid-guided nuclease exhibits both cis- and trans-cleavage activity and wherein the RNP1s are formed in partitions where different partitions comprise different gRNA1 sequences; coupling the first ribonucleoprotein complexes (RNP1s) to carrier beads in the different partitions, wherein the carrier beads in different partitions comprise different nucleic acid codes thereby forming RNP1/carrier beads; combining the RNP1/carrier beads from the partitions forming an RNP1/carrier bead library; providing a reaction mixture comprising: second ribonucleoprotein complexes (RNP2s) comprising a second nucleic acid-guided nuclease and a second gRNA that is not complementary to the target nucleic acids of interest; wherein the second nucleic acid-guided nuclease exhibits both cis- and trans-cleavage activity; a plurality of blocked nucleic acid molecules comprising a sequence complementary to the second gRNA, wherein the blocked nucleic acid molecules comprise: a first region recognized by the second gRNA of the RNP2 complex and one or more second regions not complementary to the first region forming at least one loop; and a plurality of reporter moieties comprising a detectable signal wherein the detectable signal is activated by the trans-cleavage activity of the RNP1s and/or RNP2s; providing a microfluidic droplet system comprising a main flow channel, an RNP1 introduction channel, a carrier fluid introduction channel, and a sample introduction channel; introducing a first aqueous fluid through the main flow channel, wherein the first aqueous fluid comprises the reaction mixture; introducing a second aqueous fluid through the RNP1 introduction channel into the first aqueous fluid in the main flow channel, wherein the second aqueous fluid comprises the RNP1/carrier bead library; following introduction of the second aqueous fluid into the main flow channel, introducing a carrier fluid through one or more carrier fluid introduction channels into the main flow channel thereby forming first aqueous droplets in the carrier fluid, wherein the first aqueous droplets comprise the reaction mixture and a Poisson or super Poisson distribution of RNP1/carrier beads; flowing an aqueous sample through a sample introduction into the carrier fluid thereby forming second aqueous droplets in the carrier fluid; merging the first and second aqueous droplets; providing conditions for the target nucleic acids of interest in the sample, if present, to bind to the RNP1s; and detecting fluorescence in the aqueous droplets.

In some aspects of the second, third and fourth exemplary embodiments, the method further comprises, after the step of flowing a carrier fluid through one or more carrier fluid introduction channels, the step of decoupling the nucleic acid barcodes and RNP1s from the carrier beads. In some aspects of the second, third and fourth exemplary embodiments, the carrier beads comprise agarose, acrylamide, alginate, polyethylene glycol, or chitosan. In some aspects of all exemplary embodiments, the methods further comprise the steps of sorting the droplets with detectable signal from the aqueous droplets without detectable signal; pooling the droplets with detectable signal; separating the droplets with detectable signal from carrier fluid; and sequencing the nucleic acid barcodes present in the droplets with detectable signal. In some aspects of these embodiments, the detectable signal is a fluorescent signal and the sorting step is accomplished by optical sorting. In such embodiments, the microfluidic droplet system may further comprise an optical detection device.

In some aspects of these embodiments, the carrier fluid is a non-polar hydrophobic fluid, and in some aspects, the non-polar hydrophobic fluid is a fluorinated oil.

In some aspects, the aqueous droplets comprising reaction mixture and RNP1s or reaction mixture and carrier beads in the carrier fluid have a volume of approximately 50 fL to 10 nL, and in some aspects, the aqueous droplets comprising reaction mixture and RNP1s or reaction mixture and carrier beads in the carrier fluid have a volume of approximately 1 pL to 1 nL.

In some aspects of these embodiments, the aqueous droplets comprising the reaction mixture and RNP1s or the aqueous droplets comprising the reaction mixture and the carrier beads in the carrier fluid flow through the main flow channel at a rate of approximately 10 droplets/minute to 150 droplets/minute, or approximately 10 droplets/minute to 100 droplets/minute, or approximately 25 droplets/minute to 75 droplets/minute.

In some aspects of these embodiments, there are 10 different gRNA1 sequences in the RNP1/carrier bead library (or RNP1/RNP2/carrier bead library), or 50 different gRNA1 sequences in the RNP1/carrier bead library (or RNP1/RNP2/carrier bead library), or 100 different gRNA1 sequences in the RNP1/carrier bead library (or RNP1/RNP2/carrier bead library), or 250, or 300, or 400, or 500, or 600, or 700, or 800 or 900 or 1000 or more different gRNA1 sequences in the RNP1/carrier bead library (or RNP1/RNP2/carrier bead library).

These aspects and other features and advantages of the invention are described below in more detail.

### BRIEF DESCRIPTION OF THE DRAWINGS

The foregoing and other features and advantages of the present invention will be more fully understood from the following detailed description of exemplary embodiments taken in conjunction with the accompanying drawings in which:

FIG. 1 is an overview of the general principles underlying the signal boost cascade assay described in detail herein, where target nucleic acids of interest from a sample do not need to be amplified before detection.

FIG. 2A is a diagram showing the sequence of steps in an exemplary signal boost cascade assay utilizing blocked nucleic acid molecules.

FIG. 2B is a simplified graphic showing an exemplary blocked nucleic acid molecule and a method for unblocking the blocked nucleic acid molecules of the disclosure.

FIG. 3A is a diagram showing the sequence of steps in an exemplary signal boost cascade assay utilizing circular blocked primer molecules and linear template molecules.

FIG. 3B is a diagram showing the sequence of steps in an exemplary signal boost cascade assay utilizing circular blocked primer molecules and circular template molecules.

FIG. 4 is a diagram showing the sequence of steps in an exemplary signal boost cascade assay utilizing blocked guide nucleic acid (gRNA) molecules.

FIG. 5 illustrates three exemplary embodiments of reporter moieties.

FIGS. 6A-6E are graphic depictions of five embodiments of microfluidic droplet systems and methods for performing the signal boost cascade assays described herein.

It should be understood that the drawings are not necessarily to scale, and that like reference numbers refer to like features.

### Definitions

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

All of the functionalities described in connection with one embodiment of the compositions and/or methods described herein are intended to be applicable to the additional embodiments of the compositions and/or methods except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

Note that as used herein and in the appended claims, the singular forms “a,” “an,” and “the” include plural referents unless the context clearly dictates otherwise. Thus, for example, reference to “a cell” refers to one or more cells, and reference to “a system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention. Conventional methods are used for the procedures described herein, such as those provided in the art, and demonstrated in the Examples and various general references. Unless otherwise stated, nucleic acid sequences described herein are given, when read from left to right, in the 5′ to 3′ direction. Nucleic acid sequences may be provided as DNA, as RNA, or a combination of DNA and RNA (e.g., a chimeric nucleic acid).

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both limits, ranges excluding either or both of those included limits are also included in the invention.

The term “and/or” where used herein is to be taken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “about,” as applied to one or more values of interest, refers to a value that falls within 10%, 9%, 8%, 7%, 6%, 5%, 4%, 3%, 2%, 1%, or less in either direction (greater than or less than) of a stated reference value, unless otherwise stated or otherwise evident from the context.

As used herein, the terms “binding affinity” or “dissociation constant” or “K_{d}” refer to the tendency of a molecule to bind (covalently or non-covalently) to a different molecule. A high K_{d} (which in the context of the present disclosure refers to blocked nucleic acid molecules binding to RNP2, or blocked primer molecules binding to a template molecule, or blocked guide molecules binding to a nucleic acid-guided nuclease) indicates the presence of more unbound molecules, and a low K_{d} (which in the context of the present disclosure refers to blocked nucleic acid molecules binding to RNP2, or blocked primer molecules binding to a template molecule, or blocked guide molecules binding to a nucleic acid-guided nuclease) indicates the presence of more bound molecules. In the context of the present disclosure, low K_{d} values are in a range from about 100 fM to about 1 aM or lower (e.g., 100 zM) and high K_{d} values are in the range of 100 nM-100 μM (10 mM) and thus are about 10⁵- to 10¹⁰-fold or higher as compared to low K_{d} values.

As used herein, the terms “binding domain” or “binding site” refer to a region on a protein, DNA, or RNA, to which specific molecules and/or ions (ligands) may form a covalent or non-covalent bond. By way of example, a polynucleotide sequence present on a nucleic acid molecule (e.g., a primer binding domain) may serve as a binding domain for a different nucleic acid molecule (e.g., an unblocked primer molecule). Characteristics of binding sites are chemical specificity, a measure of the types of ligands that will bond, and affinity, which is a measure of the strength of the chemical bond.

As used herein, the terms “blocked guide molecule”, “blocked guide nucleic acid”, “blocked guide RNA” and “blocked gRNA” refer to CRISPR guide nucleic acids that cannot bind to the first or second RNP complex to activate cis- or trans-cleavage. The terms “unblocked guide molecule”, “unblocked guide nucleic acid”, “unblocked guide RNA” and “unblocked gRNA” refer to a formerly blocked gRNA that can bind to the second RNP complex (RNP2) to activate trans-cleavage of additional blocked gRNAs.

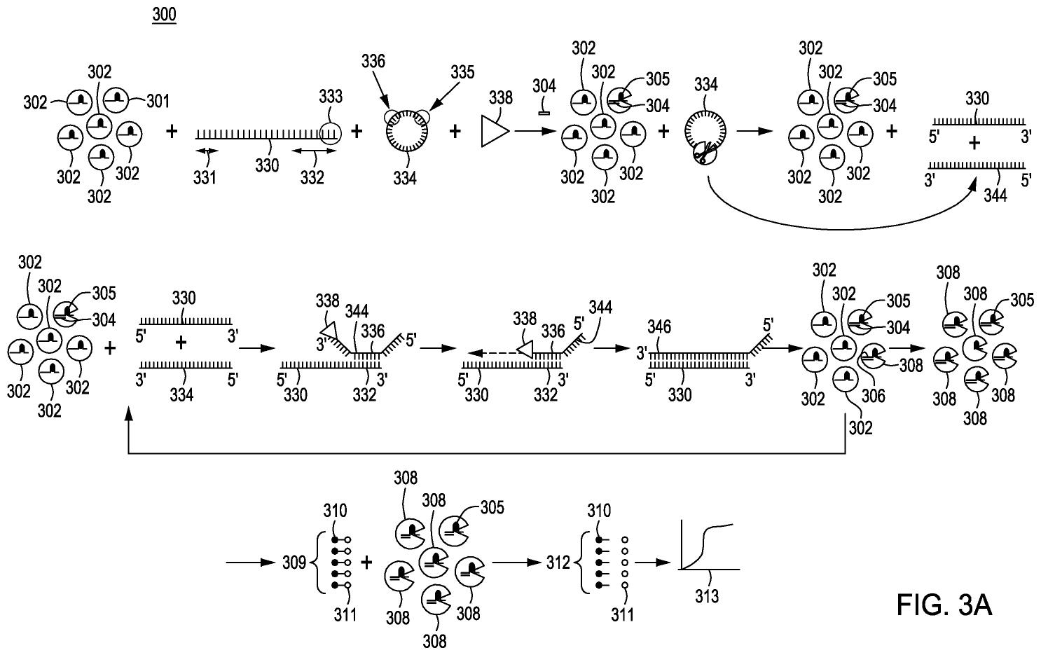

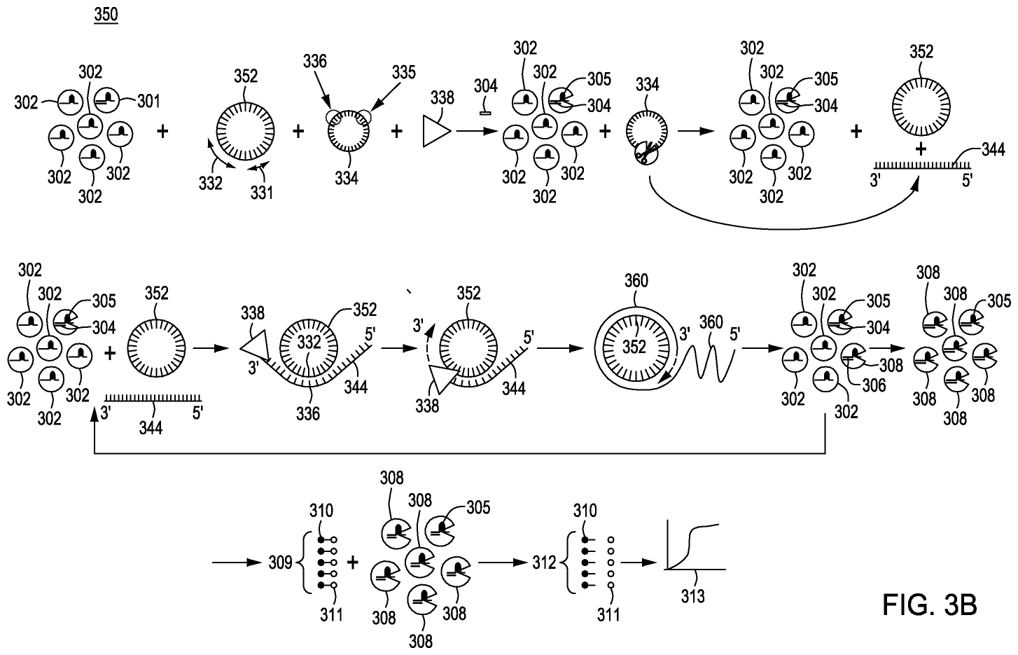

As used herein, the term “blocked nucleic acid molecule” refers to nucleic acid molecules that cannot bind to the first or second RNP complex to activate cis- or trans-cleavage. “Unblocked nucleic acid molecule” refers to a formerly blocked nucleic acid molecule that can bind to the second RNP complex (RNP2) to activate trans-cleavage of additional blocked nucleic acid molecules. A “blocked nucleic acid molecule” may be a “blocked primer molecule” in some embodiments of the cascade assay as described below in relation to FIGS. 3A and 3B.

The terms “Cas RNA-guided nucleic acid-guided nuclease” or “CRISPR nuclease” or “nucleic acid-guided nuclease” refer to a CRISPR-associated protein that is an RNA-guided nucleic acid-guided nuclease suitable for assembly with a sequence-specific gRNA to form a ribonucleoprotein (RNP) complex.

As used herein, the terms “cis-cleavage”, “cis-nucleic acid-guided nuclease activity”, “cis-mediated nucleic acid-guided nuclease activity”, “cis-nuclease activity”, “cis-mediated nuclease activity”, and variations thereof refer to sequence-specific cleavage of a target nucleic acid of interest, including an unblocked nucleic acid molecule, a synthesized activating molecule, or an RNP2 activating nucleic acid by a nucleic acid-guided nuclease in an RNP complex. Cis-cleavage is a single turn-over cleavage event in that only one substrate molecule is cleaved per event.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10, or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-ATCGAT-5′ is 100% complementary to a region of the nucleotide sequence 5′-GCTAGCTAG-3′.

As used herein, the term “contacting” refers to placement of two moieties in direct physical association, including in solid or liquid form. Contacting can occur in vitro with isolated cells (for example in a tissue culture dish or other vessel) or with reagents or in samples or in vivo by administering an agent to a subject.

A “control” is a reference standard of a known value or range of values.

The terms “guide nucleic acid” or “guide RNA” or “gRNA” refer to a polynucleotide comprising 1) a crRNA region or guide sequence capable of hybridizing to the target strand of a target nucleic acid of interest, and 2) a scaffold sequence capable of interacting or complexing with a nucleic acid-guided nuclease. The crRNA region of the gRNA is a customizable component that enables specificity in every nucleic acid-guided nuclease reaction. A gRNA can include any polynucleotide sequence having sufficient complementarity with a target nucleic acid of interest to hybridize with the target nucleic acid of interest and to direct sequence-specific binding of a ribonucleoprotein (RNP) complex containing the gRNA and nucleic acid-guided nuclease to the target nucleic acid.

“Modified” refers to a changed state or structure of a molecule. Molecules may be modified in many ways including chemically, structurally, and functionally. In one embodiment, a nucleic acid molecule (for example, a blocked nucleic acid molecule) may be modified by the introduction of non-natural nucleosides, nucleotides, and/or internucleoside linkages. In another embodiment, a modified protein (e.g., a modified or variant nucleic acid-guided nuclease) may refer to any polypeptide sequence alteration that is different from the wildtype.

As used herein, a “partition” is an isolated region (e.g., a feature surrounded by an interstitial region) or an isolated depression (e.g., a well) on a substrate, or a droplet. Partitions are used, in relation to the present disclosure, to separate a plurality of ribonucleoprotein complexes (RNP1s) comprising different guide nucleic acids (gRNA1s) into compartments (e.g., separate wells, features, or droplets). Partitions may be disposed upon a detection substrate.

The terms “percent sequence identity”, “percent identity”, or “sequence identity” refer to percent (%) sequence identity with respect to a reference polynucleotide or polypeptide sequence following alignment by standard techniques. Alignment for purposes of determining percent sequence identity can be achieved in various ways that are within the capabilities of one of skill in the art, for example, using publicly available computer software such as BLAST, BLAST-2, PSI-BLAST, or Megalign software. In some embodiments, the software is MUSCLE (Edgar, Nucleic Acids Res., 32(5):1792-1797 (2004)). Those skilled in the art can determine appropriate parameters for aligning sequences, including any algorithms needed to achieve maximal alignment over the full length of the sequences being compared. For example, in embodiments, percent sequence identity values are generated using the sequence comparison computer program BLAST (Altschul, et al., J. Mol. Biol., 215:403-410 (1990)).

As used herein, the terms “preassembled ribonucleoprotein complex”, “ribonucleoprotein complex”, “RNP complex”, or “RNP” refer to a complex containing a guide RNA (gRNA) and a nucleic acid-guided nuclease, where the gRNA is integrated with the nucleic acid-guided nuclease. The gRNA, which includes a sequence complementary to a target nucleic acid of interest, guides the RNP to the target nucleic acid of interest and hybridizes to it. The hybridized target nucleic acid/gRNA units are cleaved by the nucleic acid-guided nuclease. In the cascade assays described herein, a first ribonucleoprotein complex (RNP1) includes a first guide RNA (gRNA) specific to a target nucleic acid of interest, and a first nucleic acid-guided nuclease, such as, for example, Cas12a or Cas14a for a DNA target nucleic acid, or Cas13a for an RNA target nucleic acid. A second ribonucleoprotein complex (RNP2) used for signal boost includes a second guide RNA specific to an unblocked nucleic acid or synthesized activating molecule (or, in some embodiments, an RNP2 activating nucleic acid), and a second nucleic acid-guided nuclease, which may be different from or the same as the first nucleic acid-guided nuclease.

As used herein, the terms “protein” and “polypeptide” are used interchangeably. Proteins may or may not be made up entirely of amino acids.

As used herein, the term “sample” refers to tissues; cells or component parts; body fluids, including but not limited to peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. “Sample” may also refer to specimens or aliquots from food; agricultural products; pharmaceuticals; cosmetics; nutraceuticals; personal care products; environmental substances such as soil, water (from both natural and treatment sites), air, or sewer samples; industrial sites and products; and chemicals and compounds. A sample further may include a homogenate, lysate or extract. A sample further refers to a medium, such as a nutrient broth or gel, which may contain cellular components, such as proteins or nucleic acid molecules.

The terms “target nucleic acid of interest”, “target sequence”, “target nucleic acid molecule of interest”, “target molecule of interest”, “target nucleic acid”, or “target of interest” refer to any locus that is recognized by a gRNA sequence in vitro or in vivo. The “target strand” of a target nucleic acid of interest is the strand of the double-stranded target nucleic acid that is complementary to a gRNA. The spacer sequence of a gRNA may be 50%, 60%, 75%, 80%, 85%, 90%, 95%, 97.5%, 98%, 99% or more complementary to the target nucleic acid of interest. Optimal alignment can be determined with the use of any suitable algorithm for aligning sequences. Full complementarity is not necessarily required provided there is sufficient complementarity to cause hybridization and trans-cleavage activation of an RNP complex. A target nucleic acid of interest can include any polynucleotide, such as DNA (ssDNA or dsDNA) or RNA polynucleotides. A target nucleic acid of interest may be located in the nucleus or cytoplasm of a cell such as, for example, within an organelle of a eukaryotic cell, such as a mitochondrion or a chloroplast, or it can be exogenous to a host cell, such as a eukaryotic cell or a prokaryotic cell. The target nucleic acid of interest may be present in a sample, such as a biological or environmental sample, and it can be a viral nucleic acid molecule, a bacterial nucleic acid molecule, a fungal nucleic acid molecule, or a polynucleotide of another organism, such as a coding or a non-coding sequence, and it may include single-stranded or double-stranded DNA molecules, such as a cDNA or genomic DNA, or RNA molecules, such as mRNA, tRNA, and rRNA. The target nucleic acid of interest may be associated with a protospacer adjacent motif (PAM) sequence, which may include a 2-5 base pair sequence adjacent to the protospacer. In some embodiments 1, 2, 3, 4, 5, 6, 7, 8, 9, 10, 100, 1000, 5000 or more target nucleic acids of interest can be detected by the disclosed method.

As used herein, the terms “trans-cleavage”, “trans-nucleic acid-guided nuclease activity”, “trans-mediated nucleic acid-guided nuclease activity”, “trans-nuclease activity”, “trans-mediated nuclease activity” and variations thereof refer to indiscriminate, non-sequence-specific cleavage of a target nucleic acid molecule by a nucleic acid-guided nuclease (such as by a Cas12, Cas13, and Cas14) which is triggered by binding of N nucleotides of a target nucleic acid molecule to a gRNA and/or by cis- (sequence-specific) cleavage of a target nucleic acid molecule. Trans-cleavage is a “multiple turn-over” event, in that more than one substrate molecule is cleaved after initiation by a single turn-over cis-cleavage event.

Type V CRISPR/Cas nucleic acid-guided nucleases are a subtype of Class 2 CRISPR/Cas effector nucleases such as, but not limited to, engineered Cas12a, Cas12b, Cas12c, C2c4, C2c8, C2c5, C2c10, C2c9, CasX (Cas12e), CasY (Cas12d), Cas 13a nucleases or naturally-occurring proteins, such as a Cas12a isolated from, for example, Francisella tularensis subsp. novicida (Gene ID: 60806594), Candidatus Methanoplasma termitum (Gene ID: 24818655), Candidatus Methanomethylophilus alvus (Gene ID: 15139718), and [Eubacterium] eligens ATCC 27750 (Gene ID: 41356122), and an artificial polypeptide, such as a chimeric protein.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like.

### DETAILED DESCRIPTION

The present disclosure provides compositions of matter and methods for signal boost cascade assays that detect nucleic acids. First, the signal boost cascade assays do not require amplification of the target nucleic acids of interest yet the signal boost cascade assays retain high sensitivity. The signal boost cascade assays allow for massive multiplexing, and provide low cost, minimum workflow and results in less than ten minutes. The signal boost cascade assays described herein comprise first and second ribonucleoprotein complexes and blocked nucleic acid molecules, blocked primer molecules or blocked guide molecules. The blocked nucleic acid molecules, blocked primer molecules or blocked guide molecules keep the second ribonucleoprotein complexes (RNP2s) “locked” unless and until a target nucleic acid of interest activates the first ribonucleoprotein complexes (RNP1s). The methods comprise different variations to the steps of designing guide nucleic acids specific to several to many to a massively multiplexed number of loci in a genome and/or to several to many to a massively multiplexed number of source organisms, synthesizing first ribonucleoprotein complexes in partitions, providing signal boost cascade assay components, providing a sample, forming droplets in an immiscible carrier fluid thereby creating mini-reactors, and detecting a signal that is generated if a target nucleic acid of interest is present in the sample.

Early and accurate identification of, e.g., infectious agents, microbe contamination, variant nucleic acid sequences that indicate the presence of diseases such as cancer or contamination by heterologous sources is important in order to select correct treatment; identify tainted food, pharmaceuticals, cosmetics and other commercial goods; and to monitor the environment. Nucleic acid-guided nucleases, such as Type V nucleic acid-guided nucleases, can be utilized for the detection of target nucleic acids of interest associated with diseases, food contamination and environmental threats. However, currently available nucleic acid detection methods such as quantitative PCR (also known as real time PCR or qPCR) or CRISPR-based detection assays such as SHERLOCK™ and DETECTR™ rely on DNA amplification, which requires time and may lead to changes to the relative proportion of nucleic acids, particularly in multiplexed nucleic acid assays. The lack of rapidity for these detection assays is due to the fact that there is a significant lag phase early in the amplification process where fluorescence above background cannot be detected. With qPCR, for example, there is a lag until the cycle threshold or Ct value, which is the number of amplification cycles required for the fluorescent signal to exceed the background level of fluorescence, is achieved and can be quantified.

The present disclosure describes sample preparation and a signal boost cascade assay that can detect several to many to a massively multiplexed number of target nucleic acids of interest from several to many source organisms (e.g., DNA, RNA and/or cDNA) in a multiplexed manner in minutes (depending on the configuration of the microfluidic system and method employed) without the need for amplifying the target nucleic acid(s) of interest, thereby avoiding the drawbacks of multiplex amplification, such as primer-dimerization. The signal boost cascade assays utilize signal boost mechanisms comprising various components including nucleic acid-guided nucleases; guide RNAs (gRNAs) incorporated into ribonucleoprotein complexes (RNP complexes) (and in come embodiments, blocked guide molecules); blocked nucleic acid molecules or blocked primer molecules (or in the case of blocked guide molecules RNP2 activating nucleic acids), reporter moieties, and, in some embodiments, polymerases and template molecules. A particularly advantageous feature of the cascade assay is that, with the exception of the gRNA in RNP1 (i.e., gRNA1), the cascade assay components may be identical no matter what target nucleic acids of interest are being detected, and gRNA1 is easily programmable using known techniques and gRNA design tools known in the art.

The first two embodiments of the cascade assay provide a reaction mix comprising: a first ribonucleoprotein complex (RNP1) comprising a first Cas enzyme that exhibits both cis- and trans-cleavage activity and several to many first gRNAs; a second ribonucleoprotein complex (RNP2) comprising a second Cas enzyme that also exhibits both cis- and trans-cleavage activity and a second gRNA; either blocked nucleic acid molecules or blocked primer molecules; and reporter moieties, which may be separate molecules from the blocked nucleic acid molecules or blocked primer molecules or the reporter moieties may be incorporated into and part of the blocked nucleic acid molecules or blocked primer molecules. RNP1 is not activated unless and until a target nucleic acid of interest is detected.

The third embodiment of the cascade assay provides a reaction mix comprising: a first ribonucleoprotein complex (RNP1) comprising a first Cas enzyme that exhibits both cis- and trans-cleavage activity and several to many first gRNAs; a second Cas enzyme that also exhibits both cis- and trans-cleavage activity; blocked guide nucleic acids that, when unblocked, can form a second ribonucleotide complexes (RNP2s) with the second Cas enzyme; RNP2 activating nucleic acids; and reporter moieties. Like the first two embodiments, RNP1 is not activated unless and until a target nucleic acid of interest is detected.

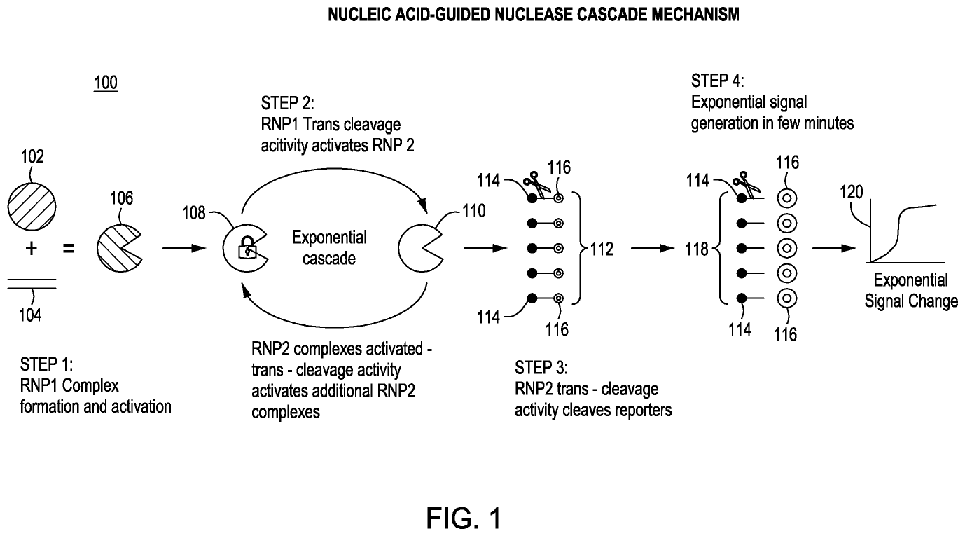

FIG. 1 provides a simplified diagram demonstrating a method (100) of a cascade assay. The cascade assay is initiated when the target nucleic acid of interest (104) binds to and activates a first pre-assembled ribonucleoprotein complex (RNP1) (102), thus forming an activated RNP2 (106). A ribonucleoprotein (RNP) complex comprises a guide RNA (gRNA) and a nucleic acid-guided nuclease, where the gRNA is integrated with the nucleic acid-guided nuclease. The gRNA, which includes a sequence complementary to the target nucleic acid of interest, guides the RNP complex to the target nucleic acid of interest and hybridizes to it. Typically, preassembled RNP complexes are employed in a reaction mix—as opposed to separate nucleic acid-guided nucleases and gRNAs—to facilitate rapid (and in the present cascade assays, virtually instantaneous) detection of the target nucleic acid(s) of interest; however, the reaction mix may comprise separate nucleic acid-guided nucleases and gRNAs.

“Activation” of RNP1 refers to activating trans-cleavage activity of the nucleic acid-guided nuclease in RNP1 (106) by binding of the target nucleic acid of interest to the gRNA of RNP1. Trans-cleavage activity if activated, as is cis-cleavage where the target nucleic acid of interest is cleaved by the nucleic acid-guided nuclease. Trans-cleavage activity (i.e., multi-turnover activity) of the nucleic acid-guided nuclease is indiscriminate, leading to non-sequence-specific cutting of nucleic acid molecules by the nucleic acid-guided nuclease of activated RNP1 (106). This trans-cleavage activity of RNP1 (106) triggers activation of blocked ribonucleoprotein complexes (RNP2s) (108) in various ways, which are described in detail below, thus, forming activated RNP2s (110). Each newly activated RNP2 (110) activates more RNP2s (108→110), which in turn cleave reporter moieties (112) to form cleaved reporter moieties (118). The reporter moieties (112) may include a synthetic molecule linked or conjugated to a quencher (114) and a fluorophore (116) such as, for example, a probe with a dye label (e.g., FAM or FITC) on the 5′ end and a quencher on the 3′ end. The quencher (114) and fluorophore (116) can be about 20-30 bases apart (or about 10-11 nm apart) or less for effective quenching via fluorescence resonance energy transfer (FRET). Reporter moieties in various configurations also are described in greater detail below.

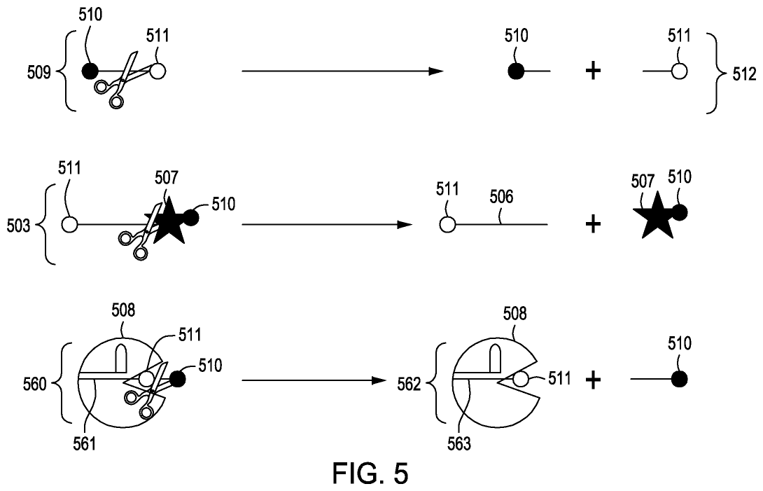

As more RNP2s are unquenched (108→110), more trans-cleavage activity is activated and more reporter moieties are unquenched; thus, the binding of the target nucleic acid of interest (104) to RNP1 (102) initiates what becomes a cascade of signal production (120), which increases exponentially; hence, the terms “signal amplification” or “signal boost.” The cascade assay thus comprises a single turnover event that triggers a multi-turnover event that then triggers another multi-turnover event in a “cascade.” As described below in relation to FIG. 5, the reporter moieties (112) may be provided as molecules that are separate from the other components of the nucleic acid-guided nuclease cascade assay, or the reporter moieties may be covalently or non-covalently linked to the blocked nucleic acid molecules or synthesized activating molecules (i.e., the target molecules for the RNP2).

Various components of the sample prep methods, cascade assay, and descriptions of how the cascade assays work are described in detail below.

### Target Nucleic Acids of Interest

The target nucleic acids of interest may comprise a DNA, RNA, or cDNA molecule. Target nucleic acids of interest may be isolated from a sample by standard laboratory techniques. The target nucleic acids of interest can originate from source organisms that are present in a sample, such as a biological sample from a subject (including non-human animals or plants), items of manufacture, or an environmental sample (e.g., water or soil). Non-limiting examples of biological samples include blood, serum, plasma, saliva, mucus, a nasal swab, a buccal swab, a cell, a cell culture, and tissue. The source of the sample could be any mammal, such as, but not limited to, a human, primate, monkey, cat, dog, mouse, pig, cow, horse, sheep, and bat, or from non-mammalian animals. Samples may also be obtained from any other source, such as air, water, soil, surfaces, food, beverages, nutraceuticals, clinical sites or products, industrial sites (including food processing sites) and products, plants and grains, cosmetics, personal care products, pharmaceuticals, medical devices, agricultural equipment and sites, and commercial samples.

In some embodiments, the target nucleic acids of interest are from one to many infectious agents (e.g., a bacteria, protozoan, insect, worm, virus, or fungus) that affect mammals, including humans. As a non-limiting example, the target nucleic acid of interest could include one or more nucleic acid molecules from bacteria, such as Bordetella parapertussis, Bordetella pertussis, Chlamydia pneumoniae, Legionella pneumophila, Mycoplasma pneumoniae, Acinetobacter calcoaceticus-baumannii complex, Bacteroides fragilis, Enterobacter cloacae complex, Escherichia coli, Klebsiella aerogenes, Klebsiella oxytoca, Klebsiella pneumoniae group, Moraxella catarrhalis, Proteus spp., Salmonella enterica, Serratia marcescens, Haemophilus influenzae, Neisseria meningitidis, Pseudomonas aeruginosa, Stenotrophomonas maltophilia, Enterococcus faecalis, Enterococcus faecium, Listeria monocytogenes, Staphylococcus aureus, Staphylococcus epidermidis, Staphylococcus lugdunensis, Streptococcus agalactiae, Streptococcus pneumoniae, Streptococcus pyogenes, Chlamydia tracomatis, Neisseria gonorrhoeae, Syphilis (Treponema pallidum), Ureaplasma urealyticum, Mycoplasma genitalium, and/or Gardnerella vaginalis. Also, as a non-limiting example, the target nucleic acid of interest could include one or more nucleic acid molecules from a virus, such as adenovirus, coronavirus HKU1, coronavirus NL63, coronavirus 229E, coronavirus OC43, severe acute respiratory syndrome coronavirus 2 (SARS-CoV-2), human metapneumovirus, human rhinovirus, enterovirus, influenza A, influenza A/H1, influenza A/H3, influenza A/H1-2009, influenza B, parainfluenza virus 1, parainfluenza virus 2, parainfluenza virus 3, parainfluenza virus 4, respiratory syncytial virus, herpes simplex virus 1, herpes simplex virus 2, human immunodeficiency virus (HIV), human papillomavirus, hepatitis A virus (HAV), hepatitis B virus (HBV), hepatitis C virus (HCV), and/or human parvovirus B19 (B19V). Also, as a non-limiting example, the target nucleic acid of interest could include one or more nucleic acid molecules from a fungus, such as Candida albicans, Candida auris, Candida glabrata, Candida krusei, Candida parapsilosis, Candida tropicalis, Cryptococcus neoformans, and/or Cryptococcus gattii. As another non-limiting example, the target nucleic acid of interest could include one or more nucleic acid molecules from a protozoan, such as Trichomonas vaginalis.

In some embodiments, other target nucleic acids of interest may be for non-infectious conditions, e.g., to be used for genotyping, including non-invasive prenatal diagnosis of, e.g., trisomies, other chromosomal abnormalities, and known genetic diseases such as Tay Sachs disease and sickle cell anemia. Other target nucleic acids of interest and samples include human biomarkers for cancer. Target nucleic acids of interest may include engineered biologics, including cells such as CAR-T cells, or target nucleic acids of interest from very small or rare samples, where only small volumes are available for testing.

The cascade assays described herein are particularly well-suited for simultaneous testing of multiple to many targets via massively multiplexed gRNAs as described below, such as from many different organisms or from many target nucleic acids of interest per locus from each source organism genome (or source chromosome or source cell or source tissue). Pools of two to 10,000 target nucleic acid of interest may be employed, e.g., pools of two to 1000, two to 100, two to 50, or two to 10 target nucleic acids of interest.

The methods described herein do not require the target nucleic acids of interest to be DNA, and in fact it is specifically contemplated that the target nucleic acid of interest may be RNA.

### Nucleic Acid-Guided Nucleases

The signal boost cascade assays comprise nucleic acid-guided nucleases in the reaction mix, either provided as a protein, a coding sequence for the protein, or, in many embodiments, in a ribonucleoprotein (RNP) complex. In some embodiments, the one or more nucleic acid-guided nucleases in the reaction mix may be, for example, a Cas nucleic acid-guided nuclease. However, any nucleic acid-guided nuclease having both cis- and trans-cleavage activity may be employed, and the same nucleic acid-guided nuclease may be used for both RNP complexes or different nucleic acid-guided nucleases may be used in RNP1 and RNP2. Typically, the nucleic acid-guided nucleases used in all RNP2s in the signal boost cascade assay are the same nucleic acid-guided nucleases. Often, however, the multiplexed signal boost cascade assay will simultaneously test for both RNA and DNA target nucleic acids. In such a case, the nucleic acid-guided nucleases in the RNP1s to detect RNA target nucleic acids of interest in a cascade assay will typically all be the same nucleic acid-guided nuclease and the nucleic acid-guided nucleases used to detect DNA target nucleic acids of interest in a cascade will typically all be the same nucleic acid-guided nuclease.

For example, RNP1 and RNP2 may both comprise Cas12a nucleic acid-guided nucleases, or RNP1 may comprise a Cas13 nucleic acid-guided nuclease and RNP2 may comprise a Cas12a nucleic acid-guided nuclease or vice versa. Note that trans-cleavage activity is not triggered unless and until cis-cleavage activity (i.e., sequence specific activity) is initiated. Nucleic acid-guided nucleases include Type V and Type VI nucleic acid-guided nucleases, as well as nucleic acid-guided nucleases that comprise a RuvC nuclease domain or a RuvC-like nuclease domain but lack an HNH nuclease domain. Nucleic acid-guided nucleases with these properties are reviewed in Makarova and Koonin, Methods Mol. Biol., 1311:47-75 (2015) and Koonin, et al., Current Opinion in Microbiology, 37:67-78 (2020) and updated databases of nucleic acid-guided nucleases and nuclease systems that include newly-discovered systems include BioGRID ORCS (orcs:thebiogrid.org); GenomeCRISPR (genomecrispr.org); Plant Genome Editing Database (plantcrispr.org) and CRISPRCasFinder (crispercas.i2bc.paris-saclay.fr).

The type of nucleic acid-guided nuclease utilized in the method of detection depends on the type of target nucleic acid of interest to be detected. For example, a DNA nucleic acid-guided nuclease (e.g., a Cas12a, Cas14a, or Cas3) should be utilized if the target nucleic acid of interest is a DNA molecule, and an RNA nucleic acid-guided nuclease (e.g., Cas13a or Cas12g) should be utilized if the target nucleic acid of interest is an RNA molecule. Exemplary nucleic acid-guided nucleases include, but are not limited to, Cas RNA-guided DNA nucleic acid-guided nucleases, such as Cas3, Cas12a (e.g., AsCas12a, LbCas12a), Cas12b, Cas12c, Cas12d, Cas12e, Cas14, Cas12h, Cas12i, and Cas12j; Cas RNA-guided RNA nucleic acid-guided nucleases, such as Cas13a (LbaCas13, LbuCas13, LwaCas13), Cas13b (e.g., CccaCas13b, PsmCas13b), and Cas12g; and any other nucleic acid (DNA, RNA, or cDNA) targeting nucleic acid-guided nuclease with cis-cleavage activity and collateral trans-cleavage activity. In some embodiments, the nucleic acid-guided nuclease is a Type V CRISPR-Cas nuclease, such as Cas12a, Cas13a, or Cas14a. In some embodiments, the nucleic acid-guided nuclease is a Type I CRISPR-Cas nuclease, such as Cas3. Type II and Type VI nucleic acid-guided nucleases may also be employed.

In an RNP with a single crRNA (i.e., lacking/without a tracrRNA), Cas12a nucleases and related homologs and orthologs interact with a PAM (protospacer adjacent motif) sequence in a target nucleic acid for dsDNA unwinding and R-loop formation. Cas12a nucleases employ a multistep mechanism to ensure accurate recognition of spacer sequences in the target nucleic acid. The WED, REC1 and PAM-interacting (PI) domains of Cas12a nucleases are responsible for PAM recognition and for initiating invasion of the crRNA in the target dsDNA and for R-loop formation. It has been hypothesized that a conserved lysine residue is inserted into the dsDNA duplex, possibly initiating template strand/non-template strand unwinding. (See Jinek, et al, Mol. Cell, 73(3):589-600.e4 (2019).) PAM binding further introduces a kink in the target strand, which further contributes to local strand separation and facilitates base paring of the target strand to the seed segment of the crRNA while the displaced non-target strand is stabilized by interactions with the PAM-interacting domains. (Id.)

The nucleic acid-guided nucleases disclosed herein include wildtype or variants of wildtype Type V nucleases LbCas12a (Lachnospriaceae bacterium Cas12a), AsCas 12a (Acidaminococcus sp. BV3L6 Cas12a), CtCas12a (Candidatus Methanoplasma termitum Cas12a), EeCas12a (Eubacterium eligens Cas12a), Mb3Cas12a (Moraxella bovoculi Cas12a), FnCas12a (Francisella novicida Cas12a), FnoCas12a (Francisella tularensis subsp. novicida FTG Cas12a), FbCas12a (Flavobacteriales bacterium Cas12a), Lb4Cas12a (Lachnospira eligens Cas12a), MbCas12a (Moraxella bovoculi Cas12a), Pb2Cas12a (Prevotella bryantii Cas12a), PgCas12a (Candidatus Parcubacteria bacterium Cas12a), AaCas12a (Acidaminococcus sp. Cas12a), BoCas12a (Bacteroidetes bacterium Cas12a), CMaCas12a (Candidatus Methanomethylophilus alvus CMx1201 Cas12a), and to-be-discovered equivalent Cas12a nucleic acid-guided nucleases and homologs and orthologs of these nucleic acid-guided nucleases (and other nucleic acid-guided nucleases that exhibit both cis-cleavage and trans-cleavage activity.

Guide RNA (gRNA)

The present disclosure detects a target nucleic acid of interest via a reaction mix containing at least two guide RNAs (gRNAs) (i.e., gRNA1 and gRNA2) each incorporated into a different RNP complex (i.e., RNP1 and RNP2, respectively). Suitable gRNAs include at least one crRNA region to enable specificity in every reaction. The gRNA1s of the RNP1s are specific to a target nucleic acid of interest and the gRNA2s of the RNP2s are specific to an unblocked nucleic acid, a synthesized activating molecule, or an RNP2 activating nucleic acid (all of which are described in detail below). As will be clear given the description below, an advantageous feature of the cascade assay is that, with the exception of the gRNA1s in the RNP1s (i.e., the gRNAs specific to the target nucleic acids of interest), the cascade assay components can stay the same (i.e., are identical or substantially identical) no matter what target nucleic acids of interest are being detected, and the gRNA1s in the RNP1s are easily reprogrammable using known techniques and gRNA design tools.

Like the nucleic acid-guided nuclease, a gRNA may be provided in the cascade assay reaction mix in a preassembled RNP, as an RNA molecule, or may also be provided as a DNA sequence to be transcribed, in, e.g., a vector backbone. Providing the gRNA in a pre-assembled RNP complex (i.e., RNP1 or RNP2) is preferred if rapid kinetics are preferred. If provided as a gRNA molecule, the gRNA sequence may include multiple endoribonuclease recognition sites (e.g., Csy4) for multiplex processing. Alternatively, if provided as a DNA sequence to be transcribed, an endoribonuclease recognition site may be encoded between neighboring gRNA sequences such that more than one gRNA can be transcribed in a single expression cassette. Direct repeats can also serve as endoribonuclease recognition sites for multiplex processing. Guide RNAs are generally about 20 nucleotides to about 300 nucleotides in length and may contain a spacer sequence containing a plurality of bases and complementary to a protospacer sequence in the target sequence. The gRNA spacer sequence may be 50%, 60%, 75%, 80%, 85%, 90%, 95%, 97.5%, 98%, 99%, or more complementary to its intended target nucleic acid of interest.

The gRNA of RNP1 is capable of complexing with the nucleic acid-guided nuclease of RNP1 to perform cis-cleavage of a target nucleic acid of interest (e.g., a DNA or RNA), which triggers non-sequence specific trans-cleavage of other molecules in the reaction mix. Guide RNAs include any polynucleotide sequence having sufficient complementarity with a target nucleic acid of interest (or target sequences generated by unblocking blocked nucleic acid molecules or target sequences generated by synthesizing synthesized activating molecules as described below). Target nucleic acids of interest (described above) preferably include a protospacer-adjacent motif (PAM), and, following gRNA binding, the nucleic acid-guided nuclease induces a double-stranded break either inside or outside the protospacer region of the target nucleic acid of interest.

In some embodiments, the gRNA (e.g., of RNP1) is an exo-resistant circular molecule that can include several DNA bases between the 5′ end and the 3′ end of a natural guide RNA and is capable of binding a target sequence. The length of the circularized guide for RNP1 can be such that the circular form of guide can be complexed with a nucleic acid-guided nuclease to form a modified RNP1 that can still retain its cis-cleavage i.e., (specific) and trans-cleavage (i.e., non-specific) nuclease activity.

In any of the foregoing embodiments, the gRNA may be a modified or non-naturally occurring nucleic acid molecule. In some embodiments, the gRNAs of the disclosure may further contain a locked nucleic acid (LNA), a bridged nucleic acid (BNA), and/or a peptide nucleic acid (PNA). By way of further example, a modified nucleic acid molecule may contain a modified or non-naturally occurring nucleoside, nucleotide, and/or internucleoside linkage, such as a 2′-O-methyl (2′-O-Me) modified nucleoside, a 2′-fluoro (2′-F) modified nucleoside, and a phosphorothioate (PS) bond, or any other nucleic acid molecule modifications described herein.

### Ribonucleoprotein (RNP) Complexes

As described above, although the cascade assay “reaction mix” or “reaction mixture” may comprise separate nucleic acid-guided nucleases and gRNAs (or coding sequences therefor), the cascade assays preferably comprise preassembled ribonucleoprotein complexes (RNPs) in the reaction mix, allowing for faster detection kinetics. The present cascade assay employs at least two types of RNP complexes—RNP1 and RNP2—each type containing a nucleic acid-guided nuclease and a gRNA. RNP1 and RNP2 may comprise the same nucleic acid-guided nuclease or may comprise different nucleic acid-guided nucleases; however, the gRNAs in RNP1 and RNP2 are different and are configured to detect different nucleic acids. In some embodiments, the reaction mixture contains about 1 fM to about 10 μM of a given RNP1, or about 1 pM to about 1 μM of a given RNP1, or about 10 pM to about 500 pM of a given RNP1. In some embodiments the reaction mixture contains about 6×10⁴ to about 6×10¹² complexes per microliter (μl) of a given RNP1, or about 6×10⁶ to about 6×10¹¹ complexes per microliter (μl) of a given RNP1. In some embodiments, the reaction mixture contains about 1 fM to about 500 μM of a given RNP2, or about 1 pM to about 250 μM of a given RNP2, or about 10 pM to about 100 μM of a given RNP2. In some embodiments the reaction mixture contains about 6×10⁴ to about 6×10¹² complexes per microliter (μl) of a given RNP2 or about 6×10⁶ to about 6×10¹² complexes per microliter (μl) of a given RNP2. See Example II below describing preassembling RNPs and Examples V and VI below describing various cascade assay conditions.

In any of the embodiments of the disclosure, the reaction mixture includes 1 to about 1,000 or more different RNP1s (e.g., 1, 2, 3, 4, 5, 6, 7, 8, 9, 10, 11, 12, 13, 14, 15, 16, 27, 28, 19, 20, 21, 22, 23, 24, 25, 50, 75, 100, 125, 150, 175, 200, 250, 300, 350, 400, 450, 500, 550, 600, 650, 700, 750, 800, 850, 900, 950, 1,0000, or 5,000 or more RNP1s), where different RNP1 s comprise a different gRNA polynucleotide sequence. For example, a reaction mixture designed for environmental or oncology testing comprises more than one unique RNP1 for the purpose of detecting more than one target nucleic acid of interest. For example, different RNP1s may be present for the purpose of targeting one target nucleic acid of interest from many sources or more than one (e.g., several to many) RNP1 s may be present for the purpose of targeting more than one target nucleic acid of interest from a single source organism (or source chromosome, source cell, source tissue, etc.).

In any of the foregoing embodiments, the gRNA1 of a specific RNP1 may be homologous or heterologous, relative to the gRNA of other RNP1(s) present in the reaction mixture. A homologous mixture of RNP1 gRNAs has a number of gRNAs with the same nucleotide sequence, whereas a heterologous mixture of RNP1 gRNAs has multiple gRNAs with different nucleotide sequences (e.g., gRNAs targeting different loci, genes, variants, and/or microbial species). As contemplated herein, there will be both homologous and heterologous gRNA1s in a given reaction; that is, there will be many RNP1s with the same gRNA1; however, there will be many RNP1s with different gRNA1 sequences. Therefore, the disclosed methods may include a reaction mixture containing more than two heterologous gRNA1s, more than three heterologous gRNA1s, more than four heterologous gRNA1s, more than five heterologous gRNA1s, more than six heterologous gRNA1s, more than seven heterologous gRNA1s, more than eight heterologous gRNA1s, more than nine heterologous gRNA1s, more than ten heterologous gRNAs, more than eleven heterologous gRNA1s, more than twelve heterologous gRNA is, more than thirteen heterologous gRNA1s, more than fourteen heterologous gRNA1s, more than fifteen heterologous gRNA1s, more than sixteen heterologous gRNA1s, more than seventeen heterologous gRNA1s, more than eighteen heterologous gRNA1s, more than nineteen heterologous gRNA1s, more than twenty heterologous gRNA1s, more than twenty-one heterologous gRNA1s, more than twenty-three heterologous gRNA1s, more than twenty-four heterologous gRNA1s, more than twenty-five heterologous gRNA1s, more than fifty heterologous gRNA1s, more than one hundred heterologous gRNA1s, more than five hundred heterologous gRNA1s, more than one thousand heterologous gRNA1s, or more than five thousand gRNA1s or more.

As a first non-limiting example of a heterologous mixture of RNP1 gRNAs, the reaction mixture may contain: a number of RNP1s (RNP1-As) having a gRNA targeting parainfluenza virus 1; a number of RNP1s (RNP1-Bs) having a gRNA targeting human metapneumovirus; a number of RNP1s (RNP1-Cs) having a gRNA targeting human rhinovirus; a number of RNP1s (RNP1-Ds) having a gRNA targeting human enterovirus; and a number of RNP1s (RNP1-Es) having a gRNA targeting coronavirus HKU1. As a second non-limiting example of a heterologous mixture of RNP1 gRNAs, the reaction mixture may contain: a number of RNP1s containing a gRNA targeting two or more SARS-Co-V-2 variants, e.g., B.1.1.7, B.1.351, P.1, B.1.617.2, BA.1, BA.2, BA.2.12.1, BA.4, and BA.5 and subvariants thereof.

As another non-limiting example of a heterologous mixture of RNP1 gRNAs, the reaction mixture may contain RNP1s targeting two or more target nucleic acids of interest from organisms that infect grapevines, such as Guignardia bidwellii (RNP1-1), Uncinula necator (RNP1-2), Botrytis cincerea (RNP1-3), Plasmopara viticola (RNP1-4), and Botryotinis fuckleina (RNP1-5).

### Reporter Moieties

The signal boost cascade assay detects a target nucleic acid of interest via detection of a signal generated in the reaction mix by a reporter moiety. In many embodiments the detection of the target nucleic acids of interest occurs within ten minutes or less. Reporter moieties can comprise DNA, RNA, a chimera of DNA and RNA, and can be single stranded, double stranded, or a moiety that is a combination of single stranded portions and double stranded portions.

Depending on the type of reporter moiety used, trans- and/or cis-cleavage by the nucleic acid-guided nuclease in RNP2 releases a signal. In some embodiments, trans-cleavage of stand-alone reporter moieties (e.g., not bound to any blocked nucleic acid molecules or blocked primer molecules) may generate signal changes at rates that are proportional to the cleavage rate, as new RNP2s are activated over time (shown in FIGS. 1C, 2A, 3A, 3B and 4 at bottom, and at top of FIG. 5). Trans-cleavage by either an activated RNP1 or an activated RNP2 may release a signal although the vast majority of trans-cleavage of the reporter moiety is due to the trans-cleavage activity of RNP2. In alternative embodiments and preferably, the reporter moiety may be bound to the blocked nucleic acid molecule, where trans-cleavage of the blocked nucleic acid molecule (or blocked primer molecule) and conversion to an unblocked nucleic acid molecule (or unblocked primer molecule) may generate signal changes at rates that are proportional to the cleavage rate as new RNP2s are activated over time, thus allowing for real time reporting of results (shown at FIG. 5, center). In yet another embodiment, the reporter moiety may be bound to a blocked nucleic acid molecule such that cis-cleavage following the binding of the RNP2 to an unblocked nucleic acid molecule releases a PAM distal sequence, which in turn generates a signal at rates that are proportional to the cleavage rate (shown at FIG. 5, bottom). In this case, activation of RNP2 by cis- (target specific) cleavage of the unblocked nucleic acid molecule directly produces a signal, rather than producing a signal via indiscriminate trans-cleavage activity. Alternatively or in addition, a reporter moiety may be bound to the gRNA.

The reporter moiety may include a synthetic molecule linked or conjugated to a reporter and quencher such as, for example, a TaqMan probe with a dye label (e.g., FAM or FITC) on the 5′ end and a quencher on the 3′ end. The reporter and quencher may be about 20-30 bases apart or less (i.e., 10-11 nm apart or less) for effective quenching via fluorescence resonance energy transfer (FRET). Alternatively, signal generation may occur through different mechanisms. Other detectable moieties, labels, or reporters can also be used to detect a target nucleic acid of interest as described herein. Reporter moieties can be labeled in a variety of ways, including direct or indirect attachment of a detectable moiety such as a fluorescent moiety, hapten, or colorimetric moiety.

Examples of detectable moieties include, but are not limited to, various radioactive moieties, enzymes, prosthetic groups, fluorescent markers, luminescent markers, bioluminescent markers, metal particles, and protein-protein binding pairs, e.g., protein-antibody binding pairs. Examples of fluorescent moieties include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, cyanines, dansyl chloride, phycocyanin, and phycoerythrin. Examples of bioluminescent markers include, but are not limited to, luciferase (e.g., bacterial, firefly, click beetle and the like), luciferin, and aequorin. Examples of enzyme systems having visually detectable signals include, but are not limited to, galactosidases, glucorinidases, phosphatases, peroxidases, and cholinesterases. Identifiable markers also include radioactive elements such as ¹²⁵I, ³⁵S, ¹⁴C, or ³H. Reporters can also include a change in pH or charge of the cascade assay reaction mix.

The methods used to detect the generated signal will depend on the reporter moiety or moieties used. For example, a radioactive label can be detected using a scintillation counter, photographic film as in autoradiography, or storage phosphor imaging. Fluorescent labels can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Enzymatic labels can be detected by providing the appropriate substrates for the enzyme and detecting the resulting reaction product. Simple colorimetric labels can be detected by observing the color associated with the label. When pairs of fluorophores are used in an assay, fluorophores are chosen that have distinct emission patterns (wavelengths) so that they can be easily distinguished.

Single-stranded, double-stranded or reporter moieties comprising both single- and double-stranded portions can be introduced to show a signal change proportional to the cleavage rate, which increases with every new activated RNP2 complex over time. In some embodiments and as described in detail below, reporter moieties can also be embedded into the blocked nucleic acid molecules (or blocked primer molecules) for real time reporting of results.

For example, the method of detecting a target nucleic acid of interest in a sample using a cascade assay as described herein can involve contacting the reaction mix with a labeled detection ssDNA containing a fluorescent resonance energy transfer (FRET) pair, a quencher/phosphor pair, or both. A FRET pair consists of a donor chromophore and an acceptor chromophore, where the acceptor chromophore may be a quencher molecule. FRET pairs (donor/acceptor) suitable for use include, but are not limited to, EDANS/fluorescein, IAEDANS/fluorescein, fluorescein/tetramethylrhodamine, fluorescein/Cy 5, IEDANS/DABCYL, fluorescein/QSY-7, fluorescein/LC Red 640, fluorescein/Cy 5.5, Texas Red/DABCYL, BODIPY/DABCYL, Lucifer yellow/DABCYL, coumarin/DABCYL, and fluorescein/LC Red 705. In addition, a fluorophore/quantum dot donor/acceptor pair can be used. EDANS is (5-((2-Aminoethyl)amino)naphthalene-1-sulfonic acid); IAEDANS is 5-({2-[(iodoacetyl)amino]ethyl}amino)naphthalene-1-sulfonic acid); DABCYL is 4-(4-dimethylaminophenyl) diazenylbenzoic acid. Useful quenchers include, but are not limited to, BHQ, DABCYL, QSY 7 and QSY 33.

In any of the foregoing embodiments, the reporter moiety may comprise one or more modified nucleic acid molecules, containing a modified nucleoside or nucleotide. In some embodiments the modified nucleoside or nucleotide is chosen from 2′-O-methyl (2′-O-Me) modified nucleoside, a 2′-fluoro (2′-F) modified nucleoside, and a phosphorothioate (PS) bond, or any other nucleic acid molecule modifications described below.

### Nucleic Acid Modifications

For any of the nucleic acid molecules described herein (e.g., blocked nucleic acid molecules, blocked primer molecules, gRNAs, template molecules, synthesized activating molecules, RNP2 activating nucleic acids and reporter moieties), the nucleic acid molecules may be used in a wholly or partially modified form. Typically, modifications to the blocked nucleic acid molecules, gRNAs, template molecules, reporter moieties, blocked primer molecules and/or blocked guide molecules described herein are introduced to optimize the molecule's biophysical properties (e.g., increasing nucleic acid-guided nuclease resistance and/or increasing thermal stability). Modifications typically are achieved by the incorporation of, for example, one or more alternative nucleosides, alternative sugar moieties, and/or alternative internucleoside linkages.

For example, one or more of the cascade assay components may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The nucleic acid molecules described herein (e.g., blocked nucleic acid molecules, blocked primer molecules, gRNAs, reporter molecules, synthesized activating molecules, and template molecules) may also include nucleobases in which the purine or pyrimidine base is replaced with other heterocycles, for example 7-deaza-adenine, 7-deazaguanosine, 2-aminopyridine, and/or 2-pyridone. Further modification of the nucleic acid molecules described herein may include nucleobases disclosed in U.S. Pat. No. 3,687,808; Kroschwitz, ed., The Concise Encyclopedia of Polymer Science and Engineering, NY, John Wiley & Sons, 1990, pp. 858-859; Englisch, et al., Angewandte Chemie, 30:613 (1991); and Sanghvi, Chapter 16, Antisense Research and Applications, CRC Press, Gait, ed., 1993, pp. 289-302.

In addition to or as an alternative to nucleoside modifications, the cascade assay components may comprise 2′ sugar modifications, including 2′-O-methyl (2′-O-Me), 2′-methoxyethoxy (2′-O—CH₂CH₂OCH₃, also known as 2′-O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other possible 2′-modifications that can modify the nucleic acid molecules described herein (i.e., blocked nucleic acid molecules, gRNAs, synthesized activating molecules, reporter molecules, and blocked primer molecules) may include all possible orientations of OH; F; O-, S-, or N-alkyl (mono- or di-); O-, S-, or N-alkenyl (mono- or di-); O-, S- or N-alkynyl (mono- or di-); or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the interfering RNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

Finally, modifications to the cascade assay components may comprise internucleoside modifications such as phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

### The Signal Boosting Cascade Assay Employing Blocked Nucleic Acid Molecules

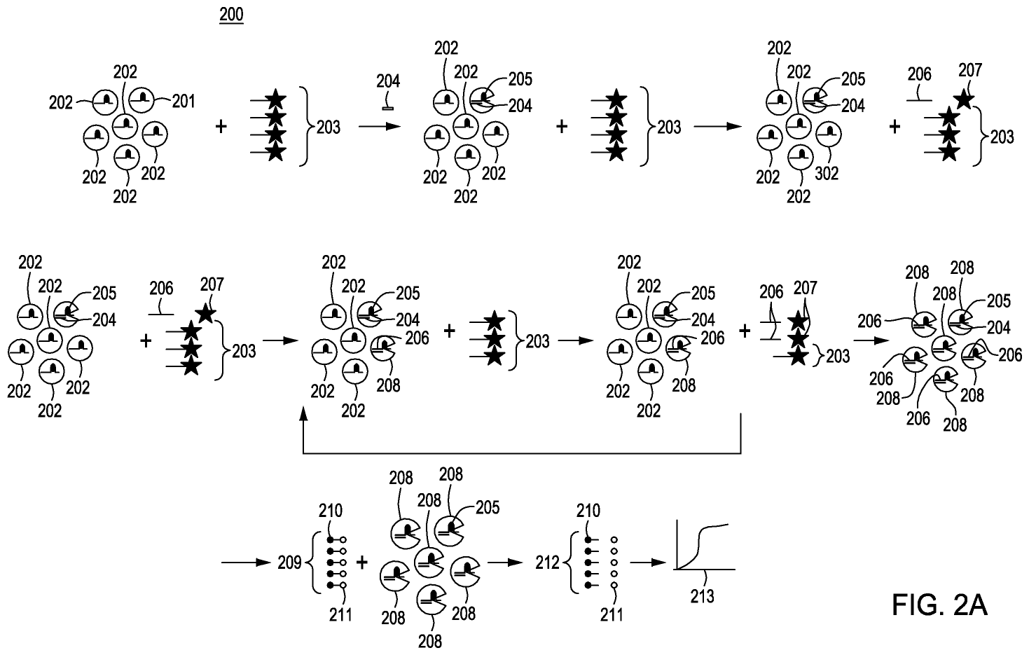

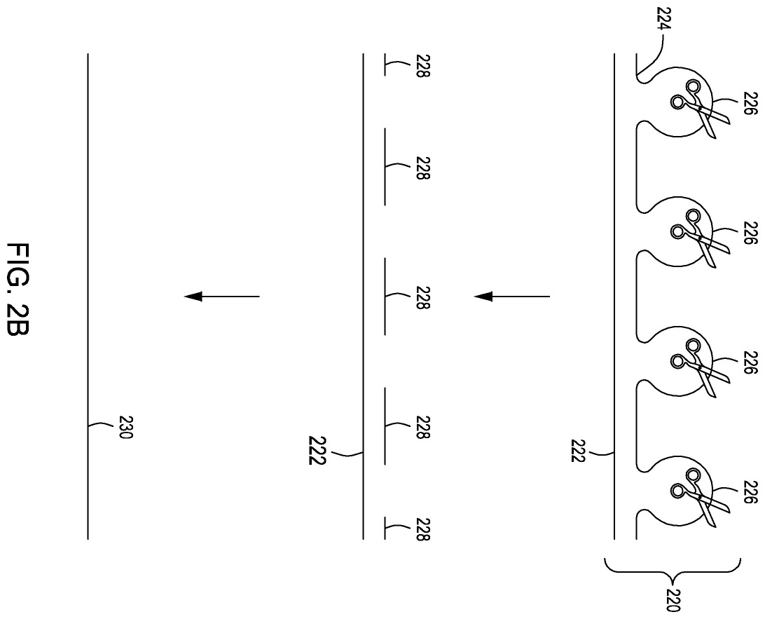

As described in detail below, the cascade assay is performed in partitions, here, droplets. FIG. 1, described above, depicts the cascade assay generally. A specific embodiment of the cascade assay utilizing blocked nucleic acid molecules is depicted in FIG. 2A and described in detail below. In this embodiment, a blocked nucleic acid molecule is used to prevent the activation of RNP2 in the absence of a target nucleic acid of interest. The method (200) in FIG. 2A begins with providing the cascade assay components RNP1 (201), RNP2 (202) and blocked nucleic acid molecules (203). RNP1 (201) comprises a gRNA specific for a target nucleic acid of interest and a nucleic acid-guided nuclease (e.g., Cas 12a or Cas 14 for a DNA target nucleic acid of interest or a Cas 13a for an RNA target nucleic acid of interest) and RNP2 (202) comprises a gRNA specific for an unblocked nucleic acid molecule and a nucleic acid-guided nuclease (again, e.g., Cas 12a or Cas 14 for a DNA unblocked nucleic acid molecule or a Cas 13a for an RNA unblocked nucleic acid molecule). As described above, the nucleic acid-guided nucleases in RNP1 (201) and RNP2 (202) can be the same or different depending on the type of target nucleic acid of interest and unblocked nucleic acid molecule. What is key, however, is that the nucleic acid-guided nucleases in RNP1 and RNP2 may be activated to have trans-cleavage activity following target nucleic acid or unblocked nucleic acid molecule binding and/or initiation of cis-cleavage activity.

In a first step (top left of FIG. 2A), a sample comprising a target nucleic acid of interest (204) is added to the signal boost cascade assay reaction mix. Keep in mind that although shown as a single reaction, the method depicted in FIG. 2A is performed with several to many to a massively multiplexed number of RNP1s. The target nucleic acid of interest (204) combines with and activates RNP1 (205) but does not interact with or activate RNP2 (202). Once activated, RNP1 binds the target nucleic acid of interest (204) initiating cleavage of the target nucleic acid of interest (204) via sequence-specific cis-cleavage, as well as activating non-specific trans-cleavage of other nucleic acids present in the reaction mix, including the blocked nucleic acid molecules (203). At least one of the blocked nucleic acid molecules (203) becomes an unblocked nucleic acid molecule (206) when the blocking moiety (207) is removed (top right of FIG. 2A). As described below, “blocking moiety” may refer to nucleoside modifications, topographical configurations such as secondary structures, and/or structural modifications.

Once at least one of the blocked nucleic acid molecules (203) is unblocked, the unblocked nucleic acid molecule (206) can then bind to and activate an RNP2 (208) (middle center of FIG. 2A). Because the nucleic acid-guided nucleases in the RNP1s (205) and RNP2s (208) have both cis- and trans-cleavage activity, the trans-cleavage activity causes more blocked nucleic acid molecules (203) become unblocked nucleic acid molecules (206) triggering activation of even more RNP2s (208) and more trans-cleavage activity in a cascade. FIG. 2A at bottom depicts the concurrent activation of reporter moieties. Intact reporter moieties (209) comprise a quencher (210) and a fluorophore (211) linked by a nucleic acid sequence. As described above in relation to FIG. 1, the reporter moieties are also subject to trans-cleavage by activated RNP1 (205) and RNP2 (208). The intact reporter moieties (209) become activated reporter moieties (212) when the quencher (210) is separated from the fluorophore (211), emitting a fluorescent signal (213). Signal strength increases rapidly as more blocked nucleic acid molecules (203) become unblocked nucleic acid molecules (206) triggering cis-cleavage activity of more RNP2s (208) and thus more trans-cleavage activity of the reporter moieties (209). Again, the reporter moieties are shown here as separate molecules from the blocked nucleic acid molecules, but other configurations may be employed and are discussed in relation to FIG. 5. As stated above, one particularly advantageous feature of the cascade assay is that, with the exception of the gRNA in the RNP1 (gRNA1), the cascade assay components are modular in the sense that the components can stay the same no matter what target nucleic acids of interest are being detected.

FIG. 2B is a diagram showing an exemplary blocked nucleic acid molecule (220) and an exemplary technique for unblocking the blocked nucleic acid molecules described herein. A blocked single-stranded or double-stranded, circular or linear, DNA or RNA molecule (or a combination of DNA and RNA) (220) comprising a target strand (222) may contain a partial hybridization with a complementary non-target strand nucleic acid molecule (224) containing unhybridized and cleavable secondary loop structures (226) (e.g., hairpin loops, tetraloops, pseudoknots, junctions, kissing hairpins, internal loops, bulges, and multibranch loops). Trans-cleavage of the loops by, e.g., activated RNP1s or RNP2s, generates short strand nucleotide sequences or regions (228) which, because of the short length and low melting temperature Tm can dehybridize at room temperature (e.g., 15°−25° C.), thereby unblocking the blocked nucleic acid molecule (220) to create an unblocked nucleic acid molecule (230), enabling the internalization of the unblocked nucleic acid molecule (230) (target strand) into an RNP2, leading to RNP2 activation.

A blocked nucleic acid molecule may be single-stranded or double-stranded, circular or linear, and may further contain a partially hybridized nucleic acid sequence containing cleavable secondary loop structures, as exemplified in FIG. 2B. Such blocked nucleic acid molecules typically have a low binding affinity, or high dissociation constant (K_{d}) in relation to binding to RNP2 and may be referred to herein as a high K_{d} nucleic acid molecule. In the context of the present disclosure, the binding of blocked or unblocked nucleic acid molecules or blocked or unblocked primer molecules to RNP2, low K_{d} values range from about 100 fM to about 1 aM or lower (e.g., 100 zM) and high K_{d} values are in the range of 100 nM to about 10-100 10 mM and thus are about 10⁵-, 10⁶-, 107-, 108-, 109- to 10¹⁰-fold or higher as compared to low K_{d} values. Of course, the ideal blocked nucleic acid molecule would have an “infinite K_{d}.”

The blocked nucleic acid molecules (high K_{d} molecules) described herein can be converted into unblocked nucleic acid molecules (low K_{d} molecules—also in relation to binding to RNP2) via cleavage of nuclease-cleavable regions (e.g., via active RNP1s and RNP2s). The unblocked nucleic acid molecule has a higher binding affinity for the gRNA in RNP2 than does the blocked nucleic acid molecule.

Once the unblocked nucleic acid molecule is bound to RNP2, the RNP2 activation triggers trans-cleavage activity, which in turn leads to more RNP2 activation by further cleaving blocked nucleic acid molecules, resulting in a positive feedback loop or cascade.

In embodiments where blocked nucleic acid molecules are linear and/or form a secondary structure, the blocked nucleic acid molecules may be single-stranded (ss) or double-stranded (ds) and contain a first nucleotide sequence and a second nucleotide sequence. The first nucleotide sequence has sufficient complementarity to hybridize to a gRNA of RNP2, and the second nucleotide sequence does not. The first and second nucleotide sequences of a blocked nucleic acid molecule may be on the same nucleic acid molecule (e.g., for single-strand embodiments) or on separate nucleic acid molecules (e.g., for double-strand embodiments). Trans-cleavage (e.g., via RNP1 or RNP2) of the second nucleotide sequence converts the blocked nucleic acid molecule to a single-strand unblocked nucleic acid molecule. The unblocked nucleic acid molecule contains only the first nucleotide sequence, which has sufficient complementarity to hybridize to the gRNA of RNP2, thereby activating the trans-cleavage activity of RNP2.

In some embodiments, the second nucleotide sequence at least partially hybridizes to the first nucleotide sequence, resulting in a secondary structure containing at least one loop (e.g., hairpin loops, tetraloops, pseudoknots, junctions, kissing hairpins, internal loops, bulges, and multibranch loops). Such loops block the nucleic acid molecule from binding or incorporating into an RNP complex thereby initiating cis- or trans-cleavage.

In some embodiments, the blocked nucleic acid molecule may contain a protospacer adjacent motif (PAM) sequence, or partial PAM sequence, positioned between the first and second nucleotide sequences, where the first sequence is 5′ to the PAM sequence, or partial PAM sequence. Inclusion of a PAM sequence may increase the reaction kinetics internalizing the unblocked nucleic acid molecule into RNP2 and thus decrease the time to detection. In other embodiments, the blocked nucleic acid molecule does not contain a PAM sequence.

In some embodiments, the blocked nucleic acid molecules (i.e., high K_{d} nucleic acid molecules in relation to binding to RNP2) of the disclosure may include a structure represented by Formula I, Formula II, Formula III, or Formula IV described in detail in U.S. Ser. Nos. 17/861,207; 17/861,208 and 17/861,209, all filed 9 Jul. 2022.

Nucleotide mismatches can be introduced in the blocked nucleic acid molecules to reduce the melting temperature (Tm) of the segment such that once the loop (L) is cleaved, the double-strand segment is unstable and dehybridizes rapidly. The percentage of nucleotide mismatches of a given segment may vary between 0% and 50%; however, the maximum number of nucleotide mismatches is limited to a number where the secondary loop structure still forms. In other words, the number of hybridized bases can be less than or equal to the length of each double-strand segment and vary based on number of mismatches introduced.

In any of the foregoing embodiments, the blocked nucleic acid molecules of the disclosure may and preferably do further contain a reporter moiety attached thereto such that cleavage of the blocked nucleic acid releases a signal from the reporter moiety. (See FIG. 5, mechanisms depicted at center and bottom.)

Also, in any of the foregoing embodiments, the blocked nucleic acid molecule may be a modified or non-naturally occurring nucleic acid molecule. In some embodiments, the blocked nucleic acid molecules of the disclosure may further contain a locked nucleic acid (LNA), a bridged nucleic acid (BNA), and/or a peptide nucleic acid (PNA). The blocked nucleic acid molecule may contain a modified or non-naturally occurring nucleoside, nucleotide, and/or internucleoside linkage, such as a 2′-O-methyl (2′-O-Me) modified nucleoside, a 2′-fluoro (2′-F) modified nucleoside, and a phosphorothioate (PS) bond, any other nucleic acid molecule modifications described above, and any combination thereof.

In some embodiments, the blocked nucleic acid molecules are circular DNAs, RNAs or chimeric (DNA-RNA) molecules, and the blocked nucleic acid molecules may include different base compositions depending on the Cas enzyme used for RNP1 and RNP2. For the circular design of blocked nucleic acid molecules, the 5′ and 3′ ends are covalently linked together. This configuration makes internalization of the blocked nucleic acid molecule into RNP2—and subsequent RNP2 activation—sterically unfavorable, thereby blocking the progression of the cascade assay. Thus, RNP2 activation (e.g., trans-cleavage activity) happens after cleavage of a portion of the blocked nucleic acid molecule followed by linearization and internalization of unblocked nucleic acid molecule into RNP2.

In some embodiments, the blocked nucleic acid molecules are topologically circular molecules with 5′ and 3′ portions hybridized to each other using DNA, RNA, LNA, BNA, or PNA bases that have a very high melting temperature (Tm). The high Tm causes the structure to effectively behave as a circular molecule even though the 5′ and 3′ ends are not covalently linked. The 5′ and 3′ ends can also have base non-naturally occurring modifications such as phosphorothioate bonds to provide increased stability.

In embodiments where the blocked nucleic acid molecules are circularized (e.g., circular or topologically circular), each blocked nucleic acid molecule includes a first region, which is a target sequence specific to the gRNA of RNP2, and a second region, which is a sequence that can be cleaved by nuclease enzymes of activated RNP1 and/or RNP2. The first region may include a nuclease-resistant nucleic acid sequence such as, for example, a phosphorothioate group or other non-naturally occurring nuclease-resistant base modifications, for protection from trans-nucleic acid-guided nuclease activity. In some embodiments, when the Cas enzyme in both RNP1 and RNP2 is Cas12a, the first region of the blocked nucleic acid molecule includes a nuclease-resistant DNA sequence, and the second region of the blocked nucleic acid molecule includes a cleavable DNA sequence. In other embodiments, when the Cas enzyme in RNP1 is Cas12a and the Cas enzyme in RNP2 is Cas13a, the first region of the blocked nucleic acid molecule includes a nuclease-resistant RNA sequence, and the second region of the blocked nucleic acid molecule includes a cleavable DNA sequence and a cleavable RNA sequence. In yet other embodiments, when the Cas enzyme in RNP1 is Cas13a and the Cas enzyme in RNP2 is Cas12a, the first region of the blocked nucleic acid molecule includes a nuclease-resistant DNA sequence, and the second region of the blocked nucleic acid molecule includes a cleavable DNA sequence and a cleavable RNA sequence. In some other embodiments, when the Cas enzyme in both RNP1 and RNP2 is Cas13a, the first region of the blocked nucleic acid molecule includes a nuclease-resistant RNA sequence, and the second region of the blocked nucleic acid molecule includes a cleavable RNA sequence.

### The Signal Boosting Cascade Assay Employing Blocked Primer Molecules

The blocked nucleic acid molecules described above may also be blocked primer molecules. Blocked primer molecules include a sequence complementary to a primer binding domain (PBD) on a template molecule (see description below in reference to FIGS. 3A and 3B) and can have the same general structures as the blocked nucleic acid molecules described above. A PBD serves as a nucleotide sequence for primer hybridization followed by primer polymerization by a polymerase. The unblocked primer molecule can bind to a template molecule at the PBD and copy the template molecule via polymerization by a polymerase.

Specific embodiments of the cascade assay which utilize blocked primer molecules and are depicted in FIGS. 3A and 3B. As with the embodiment of the cascade assay shown in FIG. 2A, keep in mind that in the massively multiplexed cascade assays described herein, the single reaction method depicted in FIGS. 3A and 3B is performed in many reactions in separate partitions in parallel, wherein each partition comprises different RNP1s. In the embodiments using blocked nucleic acid molecules described above, activation of RNP1 by binding of N nucleotides of the target nucleic acid molecules and/or cis-cleavage of the target nucleic acid molecules initiates trans-cleavage of the blocked nucleic acid molecules which were used to activate RNP2—that is, the unblocked nucleic acid molecules are a target sequence for the gRNA in RNP2. In contrast, in the embodiments using blocked primers, activation of RNP1 and trans-cleavage unblocks a blocked primer molecule that is then used to prime a template molecule for extension by a polymerase, thereby synthesizing synthesized activating molecules that are the target sequence for the gRNA in RNP2.

FIG. 3A is a diagram showing the sequence of steps in an exemplary cascade assay (300) involving circular blocked primer molecules and linear template molecules. At left of FIG. 3A is a cascade assay reaction mix comprising 1) RNP1s (301) (only one RNP1 is shown); 2) RNP2s (302); 3) linear template molecules (330) (which is the non-target strand); 4) a circular blocked primer molecule (334) (i.e., a high K_{d} molecule); and 5) a polymerase (338), such as a (D29 polymerase. The linear template molecule (330) (non-target strand) comprises a PAM sequence (331), a primer binding domain (PBD) (332) and, optionally, a nucleoside modification (333) to protect the linear template molecule (330) from 3′→5′ exonuclease activity. Blocked primer molecule (334) comprises a cleavable region (335) and a complement (336) to the PBD (332) on the linear template molecule (330).

Upon addition of a sample comprising a target nucleic acid of interest (304) (capable of complexing with the gRNA in RNP1 (301)), the target nucleic acid of interest (304) is bound with and activates RNP1 (305) but does not interact with or activate RNP2 (302). Once activated, RNP1 cuts the target nucleic acid of interest (304) via sequence specific cis-cleavage, which activates non-specific trans-cleavage of other nucleic acids present in the reaction mix, including at least one of the blocked primer molecules (334). The circular blocked primer molecule (334) (i.e., a high K_{d} molecule, where high K_{d} relates to binding to RNP2) upon cleavage becomes an unblocked linear primer molecule (344) (a low K_{d} molecule, where low K_{d} relates to binding to RNP2), which has the region (336) complementary to the PBD (332) on the linear template molecule (330) and can bind to the linear template molecule (330) (top right of FIG. 3A).

Once the unblocked linear primer molecule (344) and the linear template molecule (330) are hybridized (i.e., hybridized at the PBD (332) of the linear template molecule (330) and the PBD complement (336) on the unblocked linear primer molecule (344)), 3′→5′ exonuclease activity of the polymerase (338) removes the unhybridized single-stranded DNA at the end of the unblocked primer molecule (344) and the polymerase (338) can copy the linear template molecule (330) to produce a synthesized activating molecule (346) which is a complement of the non-target strand, and is therefore the target strand (middle center of FIG. 3A). The synthesized activating molecule (346) is capable of activating RNP2 (302→308). As described above, because the nucleic acid-guided nuclease in the RNP2 (308) complex exhibits (that is, possesses) both cis- and trans-cleavage activity, more blocked primer molecules (334) become unblocked primer molecules (344) triggering activation of more RNP2s (308) and more trans-cleavage activity in a cascade. As stated above in relation to blocked and unblocked nucleic acid molecules (both linear and circular), the unblocked primer molecule has a higher binding affinity for the gRNA in RNP2 than does the blocked primer molecule. However, an unblocked primer molecule has a substantially higher likelihood than a blocked primer molecule to hybridize with the gRNA of RNP2.

FIG. 3A at bottom depicts the concurrent activation of reporter moieties. Intact reporter moieties (309) comprise a quencher (310) and a fluorophore (311). As described above in relation to FIGS. 1 and 2A, the reporter moieties are also subject to trans-cleavage by activated RNP1 (305) and RNP2 (308). The intact reporter moieties (309) become activated reporter moieties (312) when the quencher (310) is separated from the fluorophore (311), and the fluorophore emits a fluorescent signal (313). Signal strength increases rapidly as more blocked primer molecules (334) become unblocked primer molecules (344) generating synthesized activating molecules (346) and triggering activation of more RNP2 (308) complexes and more trans-cleavage activity of the reporter moieties (309). Again, here the reporter moieties are shown as separate molecules from the blocked nucleic acid molecules, but other configurations may be employed and are discussed in relation to FIG. 5. Also, as with the cascade assay embodiment utilizing blocked nucleic acid molecules that are not blocked primers, with the exception of the gRNA in RNP1, the cascade assay components stay the same no matter what target nucleic acid(s) of interest are being detected.

FIG. 3B is a diagram showing the sequence of steps in an exemplary cascade assay (350) involving circular blocked primer molecules and circular template molecules. The cascade assay of FIG. 3B differs from that depicted in FIG. 3A by the configuration of the template molecule. Where the template molecule in FIG. 3A was linear, in FIG. 3B the template molecule is circular. At top left of FIG. 3B is a cascade assay reaction mix comprising 1) RNP1s (301) (only one RNP1 is shown); 2) RNP2s (302); 3) a circular template molecule (352) (non-target strand); 4) a circular blocked primer molecule (334); and 5) a polymerase (338), such as a (D29 polymerase. The circular template molecule (352) (non-target strand) comprises a PAM sequence (331) and a primer binding domain (PBD) (332). Blocked primer molecule (334) comprises a cleavable region (335) and a complement (336) to the PBD (332) on the circular template molecule (352).

Upon addition of a sample comprising a target nucleic acid of interest (304) (capable of complexing with the gRNA in RNP1 (301)), the target nucleic acid of interest (304) binds to and activates RNP1 (305) but does not interact with or activate RNP2 (302). Once activated, RNP1 cuts the target nucleic acid of interest (304) via sequence specific cis-cleavage, which activates non-specific trans-cleavage of other nucleic acids present in the reaction mix, including at least one of the blocked primer molecules (334). The circular blocked primer molecule (334), upon cleavage, becomes an unblocked linear primer molecule (344), which has the region (336) complementary to the PBD (332) on the circular template molecule (352) and can hybridize with the circular template molecule (352) (top right of FIG. 3B).

Once the unblocked linear primer molecule (344) and the circular template molecule (352) are hybridized (i.e., hybridized at the PBD (332) of the circular template molecule (352) and the PBD complement (336) on the unblocked linear primer molecule (344)), 3′→5′ exonuclease activity of the polymerase (338) removes the unhybridized single-stranded DNA at the 3′ end of the unblocked primer molecule (344) (middle center of FIG. 3B). The polymerase (338) can now use the circular template molecule (352) (non-target strand) to produce concatenated activating nucleic acid molecules (360) (which are concatenated target strands), which will be cleaved by the trans-cleavage activity of activated RNP1. The cleaved regions of the concatenated synthesized activating molecules (360) (target strand) are capable of activating the RNP2 (302→308) complex.

As described above, because the nucleic acid-guided nuclease in RNP2 (308) comprises both cis- and trans-cleavage activity, more blocked primer molecules (334) become unblocked primer molecules (344) triggering activation of more RNP2s (308) and more trans-cleavage activity in a cascade. FIG. 3B at bottom depicts the concurrent activation of reporter moieties. Intact reporter moieties (309) comprise a quencher (310) and a fluorophore (311). As described above in relation to FIG. 1, the reporter moieties are also subject to trans-cleavage by activated RNP1 (305) and RNP2 (308). The intact reporter moieties (309) become activated reporter moieties (312) when the quencher (310) is separated from the fluorophore (311), and the fluorescent signal (313) is unquenched and can be detected. Signal strength increases rapidly as more blocked primer molecules (334) become unblocked primer molecules (344) generating synthesized activating nucleic acid molecules and triggering activation of more RNP2s (308) and more trans-cleavage activity of the reporter moieties (309). Again, here the reporter moieties are shown as separate molecules from the blocked nucleic acid molecules, but other configurations may be employed and are discussed in relation to FIG. 5. Also note that as with the other embodiments of the cascade assay, in this embodiment, with the exception of the gRNA in RNP1, the cascade assay components optionally may stay the same no matter what target nucleic acid(s) of interest are being detected.

The polymerases used in the “blocked primer molecule” embodiments serve to polymerize a reverse complement strand of the template molecule (non-target strand) to generate a synthesized activating molecule (target strand) as described above. In some embodiments, the polymerase is a DNA polymerase, such as a BST, T4, or Therminator polymerase (New England BioLabs Inc., Ipswich MA., USA). In some embodiments, the polymerase is a Klenow fragment of a DNA polymerase. In some embodiments the polymerase is a DNA polymerase with 5′→3′ DNA polymerase activity and 3′→5′ exonuclease activity, such as a Type I, Type II, or Type III DNA polymerase. In some embodiments, the DNA polymerase, including the Phi29, T7, Q5®, Q5U®, Phusion®, OneTaq®, LongAmp®, Vent®, or Deep Vent® DNA polymerases (New England BioLabs Inc., Ipswich MA., USA), or any active portion or variant thereof. Also, a 3′ to 5′ exonuclease can be separately used if the polymerase lacks this activity.

The Signal Boosting Cascade Assay Employing Blocked Guide (gRNA) Molecules

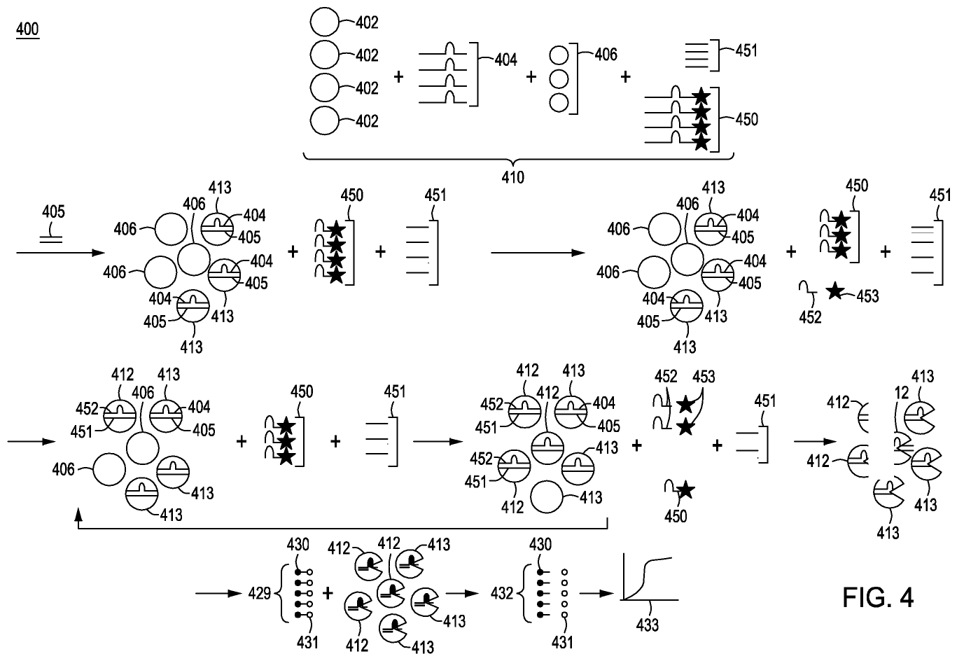

FIG. 4 is a diagram showing the sequence of steps in an exemplary cascade assay utilizing blocked guide nucleic acid (gRNA) molecules. In this embodiment, instead of a blocked nucleic acid molecule or a blocked primer molecule, a blocked guide molecule (i.e., a blocked guide RNA or blocked gRNA) is used to block activation of RNP2. The blocked guide nucleic acid molecules (blocked gRNA2s) cannot bind to and complex with the second nucleic acid nuclease to form the second ribonucleoprotein complex (RNP2) unless and until the blocked gRNA2s are unblocked via trans-cleavage activity of RNP1. The blocked gRNA2 is complementary to an RNP2 activating nucleic acid. That is, the blocked guide molecule functions like the blocked nucleic acid molecules and the blocked primer molecules to “lock” RNP2 unless and until a target nucleic acid of interest activates RNP1, the trans-cleavage activity of which then unblocks the blocked guide molecules which can then complex with the second nucleic acid-guided nuclease to form second ribonucleoprotein complexes (i.e., RNP2s) which can then be activated. As with the embodiments of the cascade assay depicted in FIGS. 2A, 3A and 3B, the massively multiplexed cascade assay depicted in FIG. 4 is performed in several to many partitions in parallel, where partitions comprise different RNP1s, though of course there may be many RNP1s with the same gRNA1s.

The cascade assay (400) in FIG. 4 begins with providing the cascade assay components in a reaction mix (410) (at top of FIG. 4) comprising: 1) first nucleic acid-guided nuclease enzymes (402); 2) first guide nucleic acids (gRNA1) (404); 3) second nucleic acid-guided nuclease enzymes (406); 4) RNP2 activating nucleic acids (451); 5) blocked guide molecules (blocked gRNA2s) (450); and 6) reporter moieties (429) (seen only at bottom of FIG. 4). The RNP2s that will be formed as a result of an activated RNP1 comprise unblocked gRNA2s that are specific for the RNP2 activating nucleic acids (451) and the second nucleic acid-guided nuclease (406) (e.g., Cas 12a or Cas 14 for DNA RNP2 activating nucleic acids or, e.g., a Cas 13a for RNA RNP2 activating nucleic acids). Both of the nucleic acid-guided nucleases that form RNP1 and RNP2 must, when activated, have trans-cleavage activity following initiation of cis-cleavage activity.

In a first step, the first Cas enzyme (402) is in the reaction mix (410) with the first guide nucleic acids (gRNA1) (404); second nucleic acid-guided nuclease (406); RNP2 activating nucleic acids (451); and blocked guide molecules (450). The first nucleic acid-guided nuclease (402) is complexed with gRNA1 (404) to form RNP1 (413) (three are shown), which then complexes with target nucleic acids of interest (405) to activate cis-cleavage of the target nucleic acids of interest (405). Also seen are second nucleic acid-guided nuclease (406) (three are shown), blocked guide molecules (blocked gRNA2s) (450) and RNP2 activating nucleic acids (451).

Once cis-cleavage of the target nucleic acids of interest (405) occurs, indiscriminate trans-cleavage activity of other nucleic acids in the reaction mix is initiated, including at least one of the blocked gRNA2s (450). The blocked gRNA2s (i.e., a high K_{d} molecules, where high K_{d} relates to binding to the second nucleic acid-guided nuclease (406)) upon cleavage become unblocked gRNA2s (452) (a low K_{d} molecule, where low K_{d} relates to binding to intact second nucleic acid-guided nuclease (406)). Thus, at least one of the blocked gRNA2s (450) becomes an unblocked gRNA2 (452) when the blocking moiety (453) is removed from the blocked gRNA2 (450) (at right of FIG. 4). As described above, “blocking moiety” may refer to nucleoside modifications, topographical configurations such as secondary structures, and/or structural modifications.

Once at least one of the blocked gRNA2s (450) is unblocked, the unblocked gRNA2 (451) can then complex with the second nucleic acid-guided nuclease (406) to form RNP2 (412) which then complexes with RNP2 activating nucleic acids (451) and cleaves the RNP2 activating nucleic acids (451) via cis-cleavage triggering trans-cleavage of more blocked gRNA2s (450) in the reaction mix (410). Because the nucleic acid-guided nucleases in the activated RNP1s (413) and RNP2s (412) have both cis- and trans-cleavage activity, the trans-cleavage activity causes more blocked gRNA2s (450) to become unblocked gRNA2s (451) triggering activation of even more RNP2s (412) and more trans-cleavage activity in a reaction cascade.

FIG. 4 at bottom depicts the concurrent activation of reporter moieties. Intact reporter moieties (429) comprise a quencher (430) and a fluorophore (431) linked by a nucleic acid sequence. As described above in relation to FIG. 1, the intact reporter moieties (429) are also subject to trans-cleavage by activated RNP1 (413) and, primarily, RNP2 (412). The intact reporter moieties (429) become unquenched reporter moieties (432) when the quencher (430) is separated from the fluorophore (431), emitting a fluorescent signal (433). Signal strength increases rapidly as more blocked gRNA2s (450) become unblocked gRNA2s (411) triggering cis-cleavage activity of more RNP2s (412) and thus more trans-cleavage activity of the reporter moieties (429). Again, the reporter moieties are shown here as separate molecules from the blocked nucleic acid molecules, but other configurations may be employed and are discussed in relation to FIG. 5. Also note that as with the other embodiments of the cascade assay, in this embodiment, with the exception of the gRNA in RNP1, the cascade assay components optionally may stay the same no matter what target nucleic acid(s) of interest are being detected.

### Reporter Moiety Configurations

FIG. 5 at top shows the mechanism discussed in relation to FIGS. 1, 2A, 3A, 3B, and 4. In this embodiment, a reporter moiety (509) is a separate molecule from the blocked nucleic acid molecules present in the reaction mix. Reporter moiety (509) comprises a quencher (510) and a fluorophore (511). An activated reporter moiety (512) emits a signal from the fluorophore (511) once it has been physically separated from the quencher (510).

FIG. 5 at center shows a blocked nucleic acid molecule (503), which is also a reporter moiety. In addition to quencher (510) and fluorophore (511), a blocking moiety (507) can be seen (see also blocked nucleic acid molecules 203 in FIG. 2A). Blocked nucleic acid molecule/reporter moiety (553) comprises a quencher (510) and a fluorophore (511). In this embodiment of the cascade assay, when the blocked nucleic acid molecule (503) is unblocked due to trans-cleavage initiated by the target nucleic acid of interest binding to RNP1, the unblocked nucleic acid molecule (506) also becomes an activated reporter moiety with fluorophore (511) separated from quencher (510). Note both the blocking moiety (507) and the quencher (510) are removed. In this embodiment, reporter signal is directly generated as the blocked nucleic acid molecules become unblocked.

FIG. 5 at the bottom shows that cis-cleavage of an unblocked nucleic acid molecule or a synthesized activating molecule at a PAM distal sequence by RNP2 generates a signal. Shown are activated RNP2 (508), unblocked nucleic acid molecule (561), quencher (510), and fluorophore (511) forming an activated RNP2 with the unblocked nucleic acid/reporter moiety intact (560). Cis-cleavage of the unblocked nucleic acid/reporter moiety (561) results in an activated RNP2 with the reporter moiety unquenched (562), comprising the activated RNP2 (508), the unblocked nucleic acid molecule with the reporter moiety activated (563), quencher (510) and fluorophore (511). Embodiments of this schema also can be used to supply the bulky modifications to the blocked nucleic acid molecules described below, and in fact a combination of the configurations of reporter moieties shown in FIG. 5 at center and at bottom may be used.

### Performing the Cascade Assay in Droplets for Massive Multiplexing

The signal boost cascade assays described herein are performed in droplets allowing for massive multiplexing and an integrated readout. Microfluidic droplet generation using two immiscible fluids (e.g., an aqueous solution and an oil) that meet at intersecting microchannels with droplets being generated at the junction between the two microfluidic channels has been known in the art for several decades. The terms “droplet” and “emulsion” are used interchangeably herein to refer to an aliquot of one fluid (here, an aqueous solution) in an immiscible carrier fluid (e.g., an oil), with the carrier fluid substantially surrounding the aqueous droplet, thereby forming a partition or “mini-reactor” in which to execute the signal boost cascade assay reactions described above. For example, in 1984 Shaw Stewart taught the use of a device to produce microfluidic emulsion droplets from two immiscible fluids that meet at an intersecting channel. (See UK Patent Application No. 2097692 to Shaw Stewart.) Shaw Stewart introduced the concept of a microfluidic “T-junction” at which droplets of an aqueous solution can be formed using a continuously flowing immiscible “carrier phase.” By the 1990s, various groups were looking to both miniaturize and automate biological and chemical reactions and by the early 2000s research groups had shown proof of principle for the use of aqueous droplets in an oil carrier for carrying out various analyses, cell sorting operations and biochemical reactions including PCR. (See, e.g., US Pub. No. 2002/0058332 to Quake, et al.)

Microfluidic droplet systems comprise microchannels of various diameters and configurations where the microchannels may be coupled to different fluid sources, receiving and/or output reservoirs, tubing, manifolds, compressors, pumps, vacuums, actuators or other flow controls. A pressure differential is used to control the flow of the fluids at the T-junction, shearing-off the aqueous fluid into the immiscible oil flow to create droplets. By adjusting the pressure of the flowing fluids, a pressure difference can be established to shear off droplets of the aqueous solution at a regular frequency as the aqueous solution enters the oil stream, thereby forming droplets in the oil stream.

The carrier fluid that is immiscible with the aqueous fluid is typically a non-polar hydrophobic fluid such as an oil, e.g., mineral oil, or an organic liquid such as hexadecane. Fluorinated oils are commonly used, with a fluorosurfactant added to stabilize the droplets that are formed. Non-limiting examples of fluorophilic components that can be used in either a surfactant and/or a continuous phase include: perfluorodecalin, perfluoromethyldecalin, perfluoroindane, perfluorotrimethyl bicyclo[3.3.1]nonane, perfluoromethyl adamantine, perfluoro-2,2,4,4-tetra-methylpentane; 9-12C perfluoro amines, e.g., perfluorotripropyl amine, perfluorotributyl amine, perfluoro-1-azatricyclic amines; bromofluorocarbon compounds, e.g., perfluorooctyl bromide and perfluorooctyl dibromide; F-4-methyl octahydroquinolidizine and perfluoro ethers, including chlorinated polyfluorocyclic ethers, perfluoro-4-methylmorpholine, perfluorotriethylamine, perfluoro-2-ethyltetrahydrofuran, perfluoro-2-butyltetrahydrofuran, perfluoropentane, perfluoro(2-methylpentane), perfluorohexane, perfluoro-4-isopropylmorpholine, perfluorodibutyl ether, perfluoroheptane, perfluorooctane, perfluorotripropylamine, perfluorononane, perfluorotributylamine, perfluorodihexyl ether, perfluoro[2-(diethylamino)ethyl-2-(N-morpholino)ethyl]ether, n-perfluorotetradecahydrophenanthrene, and mixtures thereof.

In the 2000s, a “cross junction” strategy was introduced as an alternative to the “T-juncation.” In the cross-junction approach, aqueous droplets are formed at converging flows of the immiscible fluid where a continuous phase (here, the oil phase) and the aqueous phase converging flows “pinch” off the droplets of the aqueous phase at a droplet-forming junction. This “flow focusing” approach, in which the aqueous droplet is focused by the oil phase at the cross junction, is described, for example, by Higuchi, et al. (US Pat. Pub. 2004/0068019). Microfluidic droplet devices or “chips” are available commercially through, e.g., microfluidic ChipShop™ GmbH (Jena, Germany); uFluidix™ (Toronto, Canada); Microflexis™ (Hamburg, Germany); and microLIQUID™ (Gipuzkoa, Spain). Droplet systems are available from Bio-Rad Laboratories (Hercules, CA, USA) and 10× GENOMICS® (Pleasanton, CA, USA). For exemplary microfluidic droplet systems and methods, see, e.g., U.S. Pat. Nos. 8,822,148; 8,304,193; 8,889,083; 9,216,392; 9,347,059; 9,089,844; 9,126,160; 9,500,664; 9,636,682; and 9,649,635; and US Pub. Nos. 2010/0105122; 2014/0155295; 2018/0312873; 2019/0176152; and 2019/0233878.

Performing the signal boost cascade assay in droplets is particularly useful for screening for massive numbers of target nucleic acids of interest. Allocating individual RNP1s or beads comprising RNP1s in droplets is accomplished by introducing an aqueous stream comprising the RNP1s or RNP1/carrier beads into a flowing stream of a carrier fluid such that droplets comprising the RNP1s or RNP1/carrier beads are generated at the junction of the two streams; by providing the aqueous molecule-containing stream at a certain concentration level and speed, the number of droplets containing the RNP1s or RNP1/carrier beads can be controlled. Typically, the droplet flow rate for the aqueous droplets comprising the RNP1s or RNP1/carrier beads (or RNP1/RNP2/carrier beads) in the carrier fluid is approximately 10 droplets/minute to 150 droplets/minute, or approximately 10 droplets/minute to 100 droplets/minute, or approximately 25 droplets/minute to 75 droplets/minute.

The droplets comprising the reaction mixture and RNP1s (or RNP1/carrier beads or RNP1/RNP2/carrier beads) described herein are characterized by having extremely small volumes, e.g., approximately equal to or less than 10 nL (approximately equal to or less than 5 nL, approximately equal to or less than 2 nL, approximately equal to or less than 1 nL, approximately equal to or less than 900 picoliters (pL), approximately equal to or less than 800 pL, approximately equal to or less than 700 pL, approximately equal to or less than 600 pL, approximately equal to or less than 500 pL, approximately equal to or less than 400 pL, approximately equal to or less than 300 pL, approximately equal to or less than 200 pL, approximately equal to or less than 100 pL, approximately equal to or less than 50 pL, approximately equal to or less than 25 pL, approximately equal to or less than 10 pL, approximately equal to or less than 5 pL, approximately equal to or less than 1 pL, approximately equal to or less than 500 nanoliters (fL), approximately equal to or less than 100 fL, approximately equal to or less than 50 fL, or even less.

The microfluidic droplet method allows for several to many to a massively multiplexed number of different RNP1s targeting different target nucleic acids of interest (i.e., the different “RNP1 types”) to be employed simultaneously. In most embodiments, the difference between RNP1 types is that different RNP1 types comprise different gRNA1s, where the nucleic acid-guided nuclease is the same for all RNP1 types. However, in some embodiments, different RNP1 types may comprise different nucleic acid-guided nucleases particularly the case when both DNA and RNA target nucleic acids of interest are being interrogated where, e.g., X percent of different RNP1 types comprise nucleic acid-guided nuclease A, Y percent of different RNP1 types comprise nucleic acid-guided nuclease B, and Z percent of different nucleic acid-guided nucleases comprise nucleic acid-guided nuclease C and so on, the only criterion is that the nucleic acid-guided nucleases in the RNP1s exhibit both cis- and trans-cleavage activity.

In a first type of microfluidic droplet method embodiment, the RNP1s are introduced into an aqueous stream in a “bulk” format or mode, where coded carrier beads are used to associate an RNP1 type with a nucleic acid sequence. The coded carrier beads comprise a specific nucleic acid sequence “code”, which is associated with a specific RNP1; thus, the results of the signal boost cascade assay can then be deconvolved by the sequence of the “code” from the bead. In a second type of microfluidic droplet method embodiment, different RNP1 types in an aqueous stream are introduced into the carrier fluid sequentially (i.e., a “sequential format” or mode), with blanks or “slugs” as described below used to separate the RNP1 types. In this embodiment, the results of the signal boost cascade assay can be ascertained via direct detection by the microfluidic droplet system. Note that five exemplary microfluidic droplet systems are described; however, features from any of these exemplary systems can be combined in various configurations, with additional reservoirs, channels, valves, detector devices, and the like. Note also that in the sequential format embodiments, the microfluidic droplet system can be combined on a single device (i.e., a microfluidic droplet device) with integral imaging, where in the bulk format embodiments, sequencing of nucleic acid barcodes or tags is required and the microfluidic droplet system is more likely to comprise a microfluidic droplet device comprising the reservoirs and flow channels and capable of all functions aside from sequencing, with a separate nucleic acid sequencer.

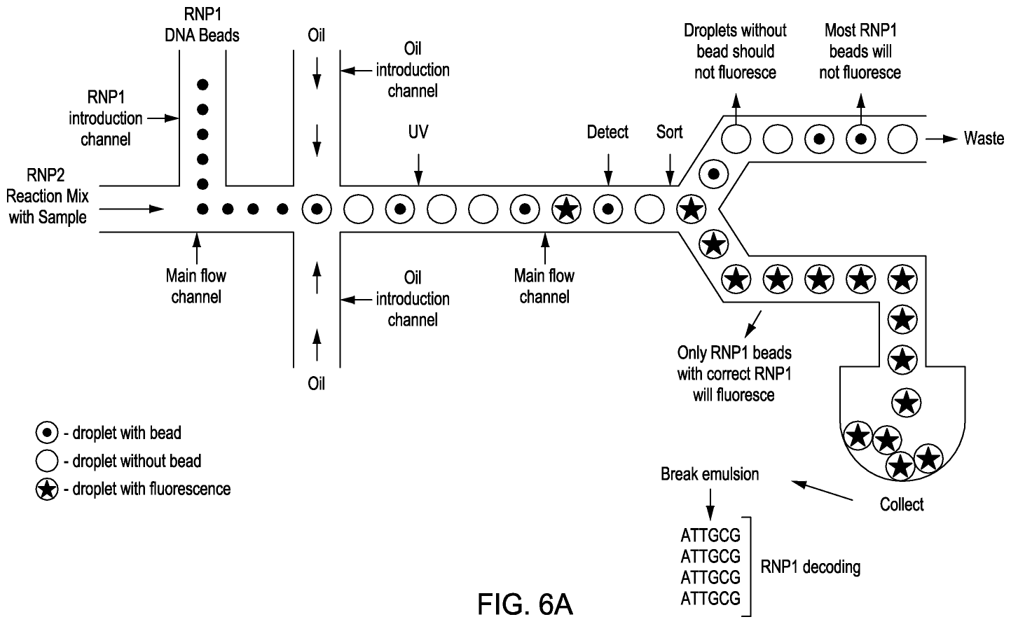

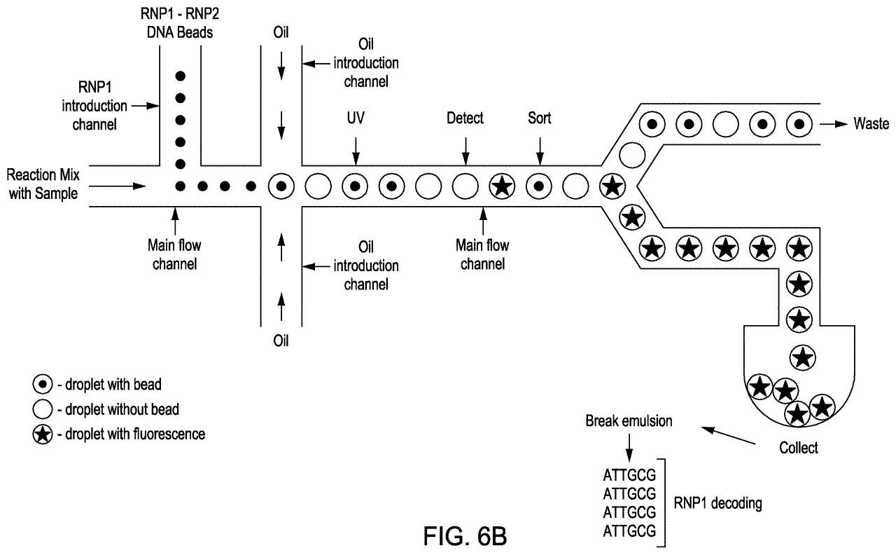

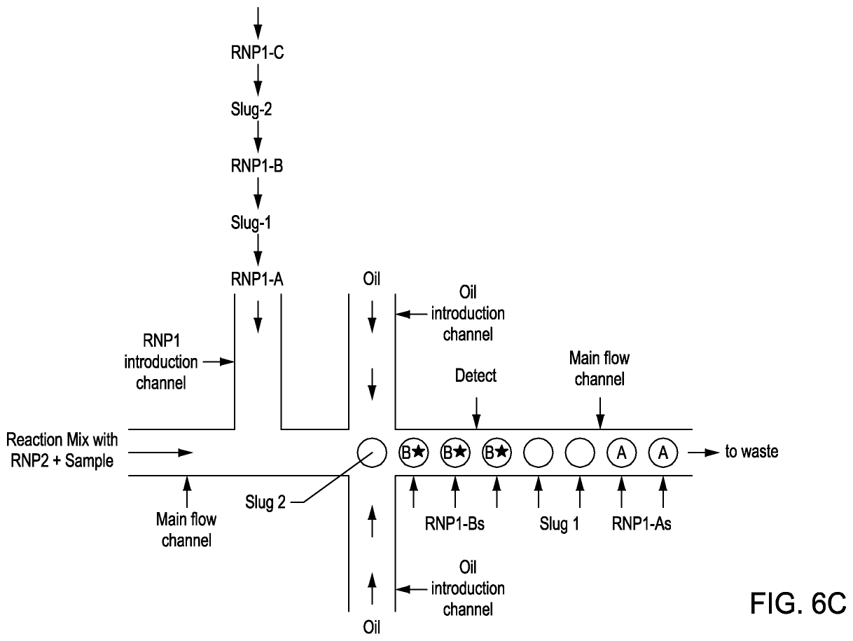

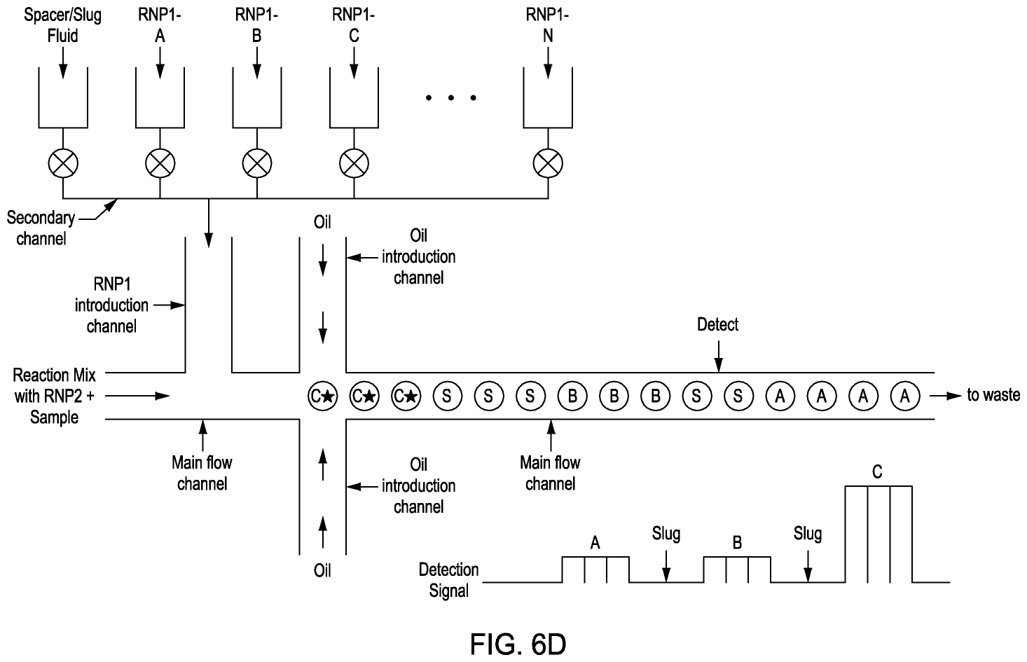

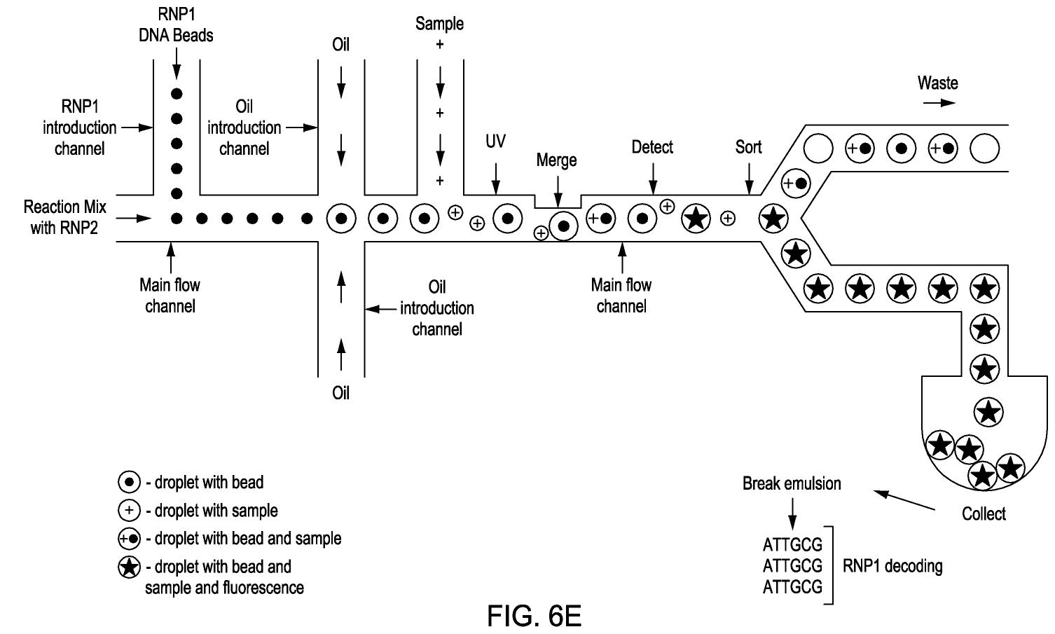

In the first type of embodiment of the present methods specifically those using carrier beads in bulk where individual beads comprise a single RNP1 type but the population of beads comprises RNP1s of many types it is desirable to control the relative flow rates of the aqueous and non-aqueous fluid(s) such that, on average, the droplets contain less than one carrier bead per droplet; that is, i.e., a Poisson distribution. A Poisson distribution ensures that any one droplet will comprise either one RNP1/carrier bead or no RNP1/carrier bead and thus one RNP1 type or no RNP1 type. In this embodiment, the droplet method allows one to partition a single bead comprising many copies of a single type of RNP1 (see FIG. 6A) or a single type of RNP1 along with RNP2s (see FIG. 6B) in a single droplet.

FIG. 6A is a representation of an exemplary microfluidic droplet system and method for performing the signal boost cascade assay in a massively multiplexed bulk format using RNP1/DNA-encoded carrier beads. The microfluidic droplet system carries out RNP1/carrier bead distribution into, e.g., picoliter-volume reagent droplets. Starting at left, an aqueous fluid comprising a reaction mixture and sample flows through a microfluidic channel. The reaction mixture will vary depending on which of the three embodiments of the signal boost cascade assay is employed; that is, whether blocked nucleic acid molecules, blocked primer molecules, or blocked guide RNAs are employed. The reaction mixture that flows in a stream from the left of FIG. 6A in the main flow channel contains all components for the reaction (here, including the sample) except for the library of carrier beads loaded with RNP1s.

To form the library of RNP1/carrier beads, RNP1s are formed in partitions, where different gRNA1s targeting different target nucleic acids of interest (i.e., the different “gRNA1 types”) are disposed into different partitions and then are complexed with a nucleic acid-guided nuclease to form RNP1s. As described above, in most embodiments the difference between “RNP1 types” is that different RNP1 types comprise different gRNA1s, where the nucleic acid-guided nuclease typically is the same for all RNP1 types (the exception being whether the gRNA1 in the RNP1 targets an RNA or DNA target nucleic acid). The different RNP1 types in the separate partitions are then coupled to DNA-encoded carrier beads in the partitions, where each DNA-encoded carrier bead is 1) optionally first linked to a photolabile (or other cleavable) linker; 2) then linked to a unique DNA sequence which serves as a “compound code” for the RNP1 type, where the compound code for each different RNP1 type is different; and 3) finally linked to the RNP1 itself.

The carrier beads employed in the methods and systems may be porous, non-porous, solid, semi-solid, semi-fluidic, fluidic, and/or a combination thereof. In some instances, a carrier bead may be dissolvable or degradable. In some embodiments, the carrier bead may be a gel bead, such as a hydrogel bead formed from molecular precursors, such as a polymeric or monomeric species. Semi-solid carrier beads may be liposomal beads, and in some embodiments, the carrier bead may be a silica bead. Beads can be rigid or may be flexible and/or compressible. A carrier bead may be of any suitable shape including, but not limited to, spherical, non-spherical, oval, oblong, amorphous, circular, cylindrical, and variations thereof. Carrier beads typically are uniform in size or nearly so. In some embodiments, the diameter of a carrier bead may be at least about 10 nanometers (nm), 100 nm, 500 nm, 1 micrometer, 5 m, 10 m, 20 m, 30 am, 40 am, 50 m, 60 m, 70 m, 80 am, 90 am, 100 am, 250 am, 500 am, 1 mm, or greater. In some cases, a bead may have a diameter of less than about 10 nm, 100 nm, 500 nm, 1 am, 5 am, 10 am, 20 am, 30 am, 40 am, 50 am, 60 am, 70 am, 80 am, 90 am, 100 am, 250 am, 500 am, 1 mm, or less.

Carrier beads may comprise natural and/or synthetic materials, such as natural polymers, synthetic polymers or both natural and synthetic polymers. Examples of natural polymers include proteins, cellulose, starch, polysaccharides, chitosan, dextran, collagen, agarose, alginic acid, alginate, acacia, agar, gelatin, shellac, xanthan gum, corn sugar gum, or guar gum. Examples of synthetic polymers include polyvinyl acetate, polyacrylamide, polyacrylate, polyethylene glycol, acrylics, nylons, silicones, spandex, viscose rayon, polycarboxylic acids, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof.

After the carrier beads are loaded with the library of RNP1 types, the RNP1/carrier beads are pooled, thus mixing the different RNP1 type carrier beads and forming a library of RNP1/carrier beads. The library of RNP1/carrier beads is then introduced into the aqueous flow of the reaction mix via the RNP1 introduction channel; here, preferably with a Poisson or super-Poisson distribution of RNP1/carrier beads in droplets. Moving right in FIG. 6A, oil (the “carrier fluid”) is then introduced from perpendicular oil introduction channels (here, above and below the main flow channel) in a “flow focusing” configuration, which “pinches off” aqueous droplets into the flow of the oil. Each aqueous droplet will comprise reaction mix, but, if the flow of the RNP1/carrier bead library and the flow of the aqueous fluid comprising the reaction mix is controlled, in some aspects only approximately half (50%) of the aqueous droplets will comprise an RNP1/carrier bead (Poisson distribution) or, in some aspects, over 90% of droplets will comprise one RNP1/carrier bead (super-Poisson distribution), or anywhere in between. By using this microfluidic approach, droplets are generated that form a reaction partition (i.e., a “mini-reactor”) for each of the RNP1/carrier beads in the signal boost cascade assay.

The aqueous droplets with RNP1-loaded beads (and without RNP1-loaded beads) proceed through the main flow channel and optionally are subjected to photochemical cleavage (“UV”) of the coded RNP1 from the carrier bead. Separating the coded RNP1 from the carrier bead separates the RNP1 coupled to the nucleic acid compound tag (i.e., code) from linkage to the carrier bead, which may facilitate binding of the RNP1 to target nucleic acids of interest in the sample, if present. The signal boost cascade assay proceeds as the droplets flow through the main flow channel.

Functionalization of the carrier beads for attachment of nucleic acid molecules (i.e., barcodes) may be achieved through a wide range of different approaches, including activation of chemical groups within a polymer, incorporation of active or activatable functional groups in the polymer structure, or attachment at the pre-polymer or monomer stage in carrier bead production. Cleavable linkers are a class of bioconjugation linker which can connect two or more molecules together and then can be cleaved once exposed to, e.g., an enzyme, photo-irradiation, or a chemical reagent. Any convenient cleavable groups may be utilized to provide for cleavage of the linker upon application of a suitable stimulus. Cleavable linkers include cleavable groups of interest, including those described by Szychowski et al., J. Am. Chem. Soc., 132:18351 (2010)), Olejnik et al., Methods in Enzymology, 291:135-154 (1998), and further described in U.S. Pat. No. 6,027,890; Olejnik et al., PNAS, 92:7590-94 (1995); Ogata et al., Anal. Chem., 74:4702-4708 (2002); Bai et al., Nucl. Acids Res., 32:535-541 (2004); Zhao et al., Anal. Chem., 74:4259-4268 (2002); Sanford et al., Chem. Mater., 10:1510-20 (1998); and linkers such as electrophilically cleavable linkers, nucleophilically cleavable linkers, photocleavable linkers, metal cleavable linkers, electrolytically-cleavable linkers, enzymatically cleavable linkers, linkers that are cleavable under reductive or oxidative conditions (e.g., a disulfide linker or a diazobenzene linker) and linkers that are cleavable using an acidic reagent (see e.g., Fauq et al., Bioconjugate Chem., 17:248-254 (2006)) or a basic reagent.

As described in the present exemplary embodiments, photocleavable (e.g., UV-cleavable, laser-cleavable, light of a particular wavelength) linkers are employed for attaching the nucleic acid barcodes and/or RNP1s or RNP2s to the carrier bead. These photocleavable linkers include photosensitive groups comprising bonds that break upon exposure to light of a certain wavelength. Suitable photocleavable linkers for use include, but are not limited to, ortho-nitrobenzyl-based linkers, phenacyl linkers, alkoxybenzoin linkers, chromium arene complex linkers, NpSSMpact linkers and pivaloylglycol linkers (see Guillier et al., Chem. Rev., 1000:2091-2157 (2000)).

In another method, disulfide bridge linkages are used between the nucleic acid barcodes and the bead, and the coded RNP1 can be released from the bead by exposing the beads to a reducing agent, including dithiothreitol (DTT) or tris(2-carboxy) phosphine (TCEP). See U.S. Pat. Nos. 9,644,204; 9,689,024; 9,951386; and 10,221,436, all assigned to 10× GENOMICS® (Pleasanton, CA, USA).

Alternatively, chemically cleavable groups include silane or —O—Si(R)₂—O—, where each R is independently selected from hydrogen, an aryl, a substituted aryl, an alkyl and a substituted alkyl, or a dialkoxydiarylsilane linker, such as a dialkoxydiphenylsilane (DADPS) linker. In yet another alternative, enzymatically cleavable groups may be utilized in the cleavable linkers. For example, the enzymatically cleavable group can be a matrix metalloproteinase cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). Another example is a thrombin cleavage site, e.g., CGLVPAGSGP (SEQ ID NO: 1). Additional suitable linkers including protease cleavage sites include linkers including one or more of the following exemplary amino acid sequences: 1) SLLKSRMVPNFN (SEQ ID NO: 2) or SLLIARRMPNFN (SEQ ID NO: 3), cleaved by cathepsin B; SKLVQASASGVN (SEQ ID NO: 4) or SSYLKASDAPDN (SEQ ID NO: 5), cleaved by an Epstein-Barr virus protease. Another method for triggering cleavage includes raising the temperature of the aqueous droplets in the carrier fluid.

Moving right in FIG. 6A, if the RNP1 is activated by a target nucleic acid of interest binding to the gRNA, both cis-cleavage of the target nucleic acid of interest occurs and indiscriminate trans-cleavage activity of the nucleic acid-guided nuclease component of the RNP1 is activated thus cleaving other nucleic acids in the reaction mixture. As described above, the other nucleic acids in the reaction mixture will differ depending on the embodiment of the signal boost cascade assay used, but will include either blocked nucleic acid molecules, blocked primer molecules, or blocked guide molecules (collectively “blocked molecules”). Unblocking of blocked molecules then initiates activation of RNP2 and the attendant cis-cleavage activity and trans-cleavage activity, which in turn initiates cleavage of reporter moieties in the reaction mixture in a cascade. In many embodiments, the cascade reaction will take place as the droplets travel through the main channel of the microfluidic droplet system; however in alternative embodiments, the main channel of the microfluidic droplet system may widen to create a sort of “reaction chamber” or “incubation chamber” that may be kept at a different temperature than other portions of the main channel or other secondary channels of the microfluidic droplet system and that, due to widening, retains the droplets for a period of time such that the cascade reactions can take place.

After the RNP1s react with target nucleic acids of interest from the sample in the reaction mixture, the aqueous droplets continue to flow through the microfluidic droplet system. Additional oil (not shown) optionally may be flowed into the existing oil flow to regulate the space between droplets (now containing completed signal boost cascade assay reactions), and a detector is used to detect the level of fluorescence in each droplet, which is generated by the RNP2 signal boost as described above in relation to FIGS. 2A, 3A, 3B and 4. The detector can be any device or method for interrogating a droplet as it passes through a detection region. In the present signal boost cascade assays using, e.g., a fluorescent reporter, one detector of particular use is an optical detector, such as a microscope or spectrophotometer and measured by a photo multiplier tube or other image processing or enhancement device, which may be coupled to a computer to digitize the signal may control the flow of the droplets via, e.g., valve action or an electroosmotic potential.

The droplets are detected, analyzed and sorted based on the intensity of a signal detected as the droplets pass through a detection region or window. Droplets having a level of reporter below a selected threshold or within a selected range are diverted into a predetermined outlet or reservoir. Active droplet sorting takes place and generally employs external fields (e.g., acoustic, electric, magnetic, and optical) to impose forces to displace the droplets for sorting. Droplet sorting in microfluidic droplet systems includes label-based sorting (here, label-based sorting of reporter moieties), detectable by optical sorting; electrokinetic mechanisms such as, e.g., electrophoresis and dielectric phoresis; acoutstrophoresis; mechanical systems; and optical switching mechanisms. For sorting, the droplets may be sorted while suspended in the oil carrier or, optionally, the droplets in the oil carrier may be further dispersed in an aqueous phase-creating a double emulsion for sorting. The optical signal is collected and projected onto a cathode of a photomultiplier tube. Optionally, part of the light may be directed onto a charge-coupled device (CCD) camera for imaging. As the droplets pass by the spectrophotometer detection window, the droplets are directed to conduits that lead to the reservoirs that collect fluorescing droplets and empty droplets depending on, e.g., acoustic, electric, magnetic, and optical fields to impose forces to displace droplets for sorting.

Following sorting of the fluorescent-positive droplets into, e.g., a microtube or other container, the emulsion (aqueous droplets in oil) is broken and the aqueous phase is collected, thereby pooling the RNP1s and the nucleic acid barcodes that bound target nucleic acids of interest that are present in the droplets collected. The identity of the RNP1 can be determined by sequencing the nucleic acid barcodes in the pooled aqueous phase (here, sequence ATTGCG), which in turn identifies the target nucleic acid(s) of interest present in the sample. See MacConnell, et al., ACS Comb. Sci., 19:181-92 (2017) for a description of functional screens using DNA-encoded compound beads in a microfluidic droplet system.

FIG. 6B is a representation of a second exemplary microfluidic droplet system and method for performing the signal boost cascade assay in a massively multiplexed bulk format using RNP1/RNP2/nucleic acid-encoded carrier beads. As with the bulk format embodiment shown in FIG. 6A and described above, the microfluidic droplet system carries out RNP1/RNP2/carrier bead distribution into, e.g., picoliter-sized reagent droplets. Starting at left, an aqueous fluid comprising a reaction mixture and sample flows through a microfluidic main flow channel. As with the embodiment described above and in relation to FIG. 6A, the reaction mixture will vary depending on which of the three embodiments of the signal boost cascade assay is employed; that is, the reaction mixture will vary by which type or configuration of blocked molecule is used (i.e., blocked nucleic acid molecule, blocked primer molecule, or blocked guide molecule). The reaction mixture that flows in a stream from the left of FIG. 6B contains all components for the reaction (here, including the sample) except for the library of carrier beads loaded with RNP1s and RNP2s.

To form the library of RNP1/RNP2/carrier beads, RNP1s are formed in partitions as described above, where different gRNA1s targeting different target nucleic acids of interest are disposed into different partitions and then RNP1s comprising these gRNA1s are formed in the partitions. As described above, in most embodiments the difference between RNP1 types is that different RNP1 types comprise different gRNA1s, where the nucleic acid-guided nuclease typically only differs depending on whether RNA or DNA target nucleic acids are being detected. In this embodiment, in addition to RNP1s being coupled to the carrier beads, the carrier beads also comprise RNP2s. As described above, the RNP2s in a reaction are typically identical; that is, all RNP2s in a reaction will have the same gRNA and nucleic acid-guided nuclease molecules. The different RNP1 types and the RNP2s in the separate partitions are then coupled to nucleic acid-encoded carrier beads in the partitions, where each carrier bead is 1) optionally first linked to a photolabile (or other cleavable) linker; 2) then linked to a unique nucleic acid sequence which serves as a “compound code” for the RNP1 type, where the compound code for each different RNP1 type is different; and 3) finally linked to the RNP1 and RNP2s. The ratio of RNP1s to RNP2s on each carrier bead can be controlled by adjusting the ratio of RNP1s and RNP2s in the partitions.

After the carrier beads are loaded with a member of the library of RNP1 types and the RNP2s, the RNP1/RNP2/carrier beads (i.e., the library of RNP1/RNP2/carrier beads) are pooled, thus mixing the different RNP1 type carrier beads. The library of RNP1/RNP2/carrier beads are then introduced via the RNP1 introduction channel into the aqueous flow of the reaction mixture in the main flow channel; here, a Poisson distribution of RNP1/RNP2/carrier beads in the droplets is preferred. Moving right in FIG. 6B, oil (the “carrier fluid”) is then introduced from perpendicular oil introduction channels (here, above and below the main flow channel) in a “flow focusing” configuration, which “pinches off” aqueous droplets into the flow of the oil. Each aqueous droplet will comprise reaction mixture, but, if the flow of the RNP1/RNP2/carrier bead library and the flow of the aqueous fluid comprising the reaction mixture is controlled, only approximately half (Poisson) to 90% (super Poisson) of the aqueous droplets will comprise an RNP1/RNP2/carrier bead. By using this microfluidic approach, droplets are generated that form a reaction partition (i.e., a “mini-reactor”) for each of the RNP1/RNP2/carrier beads in the signal boost cascade assay.

As with the embodiment described in relation to FIG. 6A, the aqueous droplets with RNP1/RNP2-loaded beads and without RNP1/RNP2-loaded beads proceed through the flow channel and are optionally subjected to, e.g., photochemical cleavage via exposure to UV light, which cleaves the coded RNP1s and RNP2s from the carrier bead. Separating the coded RNP1s and coded RNP2s from the carrier bead separates the RNPs from linkage to the carrier bead, thereby possibly facilitating the RNP1 binding to target nucleic acids of interest in the sample, if present, and freeing the RNP2s to freely bind the unblocked blocked nucleic acid molecules, if present.

Continuing to more right in FIG. 6B, if RNP1 is activated by a target nucleic acid of interest binding to the gRNA1, both cis-cleavage of the target nucleic acid of interest occurs and indiscriminate trans-cleavage activity of the nucleic acid-guided nuclease component of the RNP1 is activated thereby cleaving other nucleic acids (specifically blocked molecules) in the reaction mixture.

After the RNP1s and RNP2s are cleaved from the carrier beads and the RNP1s react with target nucleic acids of interest from the sample in the reaction mixture (if present), the aqueous droplets continue to flow through the microfluidic droplet system. Additional oil (not shown) is optionally flowed into the existing oil flow to regulate the space between droplets (now containing completed signal boost cascade assay reactions), and a detector is used to detect the level of fluorescence in each droplet. Again, the detector can be any appropriate device or method—depending on the reporter moiety or system used—for interrogating a droplet as it passes through a detection region. In the present signal boost cascade assays using, e.g., a fluorescent reporter, one detector of particular use is an optical detector, such as a microscope or spectrophotometer measured by a photo multiplier tube or other image processing or enhancement device, which may be coupled to a computer to digitize the signal may control the flow of the droplets via, e.g., valve action or an electroosmotic potential.

The droplets are detected, analyzed and sorted based on the intensity of a signal detected as the droplets pass through a detection region or window. Droplets having a level of reporter below a selected threshold or within a selected range are diverted into a predetermined outlet or reservoir. As described above, a droplet-sorting device may comprise a spectrophotometer where the fluorescent intensity of each droplet is read as it passes by the light beam. The optical signal is collected and projected onto a cathode of a photomultiplier tube. Optionally, part of the light may be directed onto a charge-coupled device (CCD) camera for imaging. As the droplets pass by the spectrophotometer detection window, the droplets are directed to the different conduits that lead to the reservoirs that collect fluorescing droplets on one hand and non-fluorescing and empty droplets on the other hand depending on, e.g., acoustic, electric, magnetic, and optical fields imposing forces to displace droplets for sorting.

Following sorting of the fluorescent-positive droplets into, e.g., a microtube or other container, the emulsion (aqueous droplets in oil) is broken and the aqueous phase collected thereby pooling the RNP1s that bound a target nucleic acid of interest, the RNP2s, and the nucleic acid barcodes that are present in the droplet. The identity of the RNP1 can be determined by sequencing the nucleic acid barcodes in the pooled reaction mixture (here, e.g., ATTGCG), which in turn identifies the target nucleic acid(s) of interest present in the sample. Again, see MacConnell, et al., ACS Comb. Sci., 19:181-92 (2017) for a description of functional screens using DNA-encoded compound beads in a microfluidic droplet system.

FIG. 6C is a representation of a third exemplary microfluidic droplet system and method for performing the signal boost cascade assay in a massively multiplexed format; however, in this embodiment, the different RNP1 types are added sequentially to the aqueous stream i.e., in a “sequential format” microfluidic droplet method as opposed to the “bulk format” microfluidic droplet method. As an alternative to the bulk format embodiment shown in FIGS. 6A and 6B and described above where carrier beads are utilized, here the sequence of, e.g., picoliter-sized droplets in the microfluidic droplet system is controlled. Starting at left, an aqueous fluid comprising a reaction mixture and sample flows through a microfluidic main flow channel. As with the embodiments described above and in relation to FIGS. 6A and 6B, the reaction mixture will vary depending on which of the three embodiments of the signal boost cascade assay is being employed; that is, the reaction mixture will vary by which type or configuration of blocked molecule is used (i.e., blocked nucleic acid molecule, blocked primer molecule, or blocked guide molecule). The reaction mixture that flows in a stream from the left of FIG. 6C contains all components for the reaction (here, including the sample and RNP2s) except for the different RNP1 types. Note, however, that in an alternative embodiment, e.g., the RNP2s may instead be combined with the different RNP1 types and delivered sequentially along with the RNP1 types.

In this sequential format embodiment, the different RNP1 types are kept separate before and after introduction into the reaction mixture flow. In this embodiment, different RNP1 types are introduced into a channel in a “burst” mode, with each different RNP1 type being introduced in a burst or packet controlled by, e.g., valves connected to the reservoirs of the RNP1s. In this embodiment, a first reservoir is opened where RNP1-A flows into the RNP1 introduction channel, where this RNP1 introduction channel then meets and flows into the main flow channel through which the reaction mixture is flowing. As with FIGS. 6A and 6B, after the RNP1-A burst liquid meets and combines in the channel with the reaction mixture, oil (the “carrier fluid”) is then introduced from perpendicular oil introduction channels (here, above and below the main flow channel) in a “flow focusing” configuration, which “pinches off” the aqueous droplets into the flow of the oil. Each aqueous droplet will comprise reaction mixture and some volume of the RNP1-A packet.

Again, droplets are generated that form a reaction partition (i.e., a “mini-reactor”) for the RNP1-As in the signal boost cascade assay. In this embodiment, unlike those described in relation to FIGS. 6A and 6B, it is not necessary for the droplets generated to comprise a Poisson or super-Poisson distribution of RNP1/carrier beads. In fact it is preferred that a number of RNP1s (e.g., RNP1-As) are present in each droplet and that there are several to many droplets generated for each RNP1 type, which aids in the statistical likelihood of positively identifying a target nucleic acid of interest present in the sample. The number of RNP1s present in each droplet and the number of droplets present for, e.g., RNP1-A, can be controlled by the concentration and volume of the RNP1-A burst and the pressure of the reaction mixture and RNP1-A burst flow. In this way, an entire reservoir of the RNP1-A burst can be used before moving on to a second reservoir with, e.g., an RNP1-B burst.

The different RNP1 type bursts are preferably separated by a “slug” or volume of separation fluid that does not comprise an RNP1. The slug serves to clear any remnants of, e.g., RNP1-A from the RNP1 introduction channel and to space out the different RNP1 types (RNP1-A, RNP1-B, RNP1-C, . . . RNP1-N) in the main flow channel and as the droplets are detected by the detector. The slug or separation fluid can be an aqueous fluid or a carrier fluid. For example, to minimize the amount of reaction mixture used, valve timing can be used to both stop the introduction of reaction mixture and start the introduction of the slug of separation fluid, which may be a carrier fluid. In another embodiment, the separation fluid may be an aqueous fluid comprising, e.g., a colored or other detectable compound that can be detected and the separation identified by the detector. Note, too, that although the RNP1-A volumes in this embodiment comprise RNP1-A only, the present embodiment could also be applied to RNP1-carrier beads; that is, instead of delivering RNP1-As in solution, batches or bursts of RNP1-A/carrier beads in solution (or RNP1-A/RNP2/carrier beads) could be employed. Further, RNP2s can be distributed into droplets by being included in the RNP1 reservoirs.

As with the embodiments described in relation to FIGS. 6A and 6B, the aqueous droplets with RNP1s (and without RNP1s (slugs)) proceed through the main flow channel. If an RNP1 is activated by a target nucleic acid of interest binding to the gRNA 1 of the RNP1, both cis-cleavage of the target nucleic acid of interest occurs and indiscriminate trans-cleavage activity of the nucleic acid-guided nuclease component of the RNP1 is activated thereby cleaving other nucleic acids (specifically blocked molecules) in the reaction mixture. Here, a target nucleic acid of interest is detected by RNP1-B (denoted by stars). As the droplets comprising the RNP1-A pass the detector, no fluorescence is detected. Additionally, as the droplets comprising the slug or separation fluid pass the detector, no fluorescence is detected. However, as the droplets comprising the activated RNP1-B (and hence activated reporter) pass the detector, fluorescence is detected and RNP-B is identified as being activated, and hence the target nucleic acid of interest for RNP1-B is identified. (See also FIG. 6D and the description therefor.)

As described above, the detector can be any device or method appropriate for detecting the reporter employed for interrogating a droplet as it passes through a detection region. In the present signal boost cascade assays using, e.g., a fluorescent reporter, one detector often employed is an optical detector, such as a microscope or spectrophotometer and measured by a photo multiplier tube or other image processing or enhancement device, which may be coupled to a computer to digitize the signal may control the flow of the droplets via, e.g., valve action or an electroosmotic potential. Note in this “sequential format” embodiment of the microfluidic droplet methods, sorting of the droplets is not necessary, as the timing of detected droplets identifies the RNP1 that has been activated, and hence the target nucleic acid(s) present.

FIG. 6D is a representation of a fourth exemplary microfluidic droplet system and method for performing the signal boost cascade assay in a massively multiplexed format, where, like the embodiment shown in FIG. 6C, the different RNP1 types are added sequentially to the aqueous stream (i.e., a “sequential format” as opposed to the “bulk format”). This embodiment is also an alternative to the bulk format embodiment shown in FIGS. 6A and 6B and described above. Like the embodiment described in relation to FIG. 6C, the sequence of, e.g., picoliter-sized droplets in the microfluidic droplet system is controlled, here by a series of reservoirs fluidly connected to the RNP1 introduction channel. Starting at left, an aqueous fluid comprising a reaction mixture including RNP2s and sample flows through a microfluidic main flow channel. As with the embodiments described above and in relation to FIGS. 6A, 6B, and 6C, the reaction mixture will vary depending on which of the three embodiments of the signal boost cascade assay is being employed; that is, the reaction mixture will vary by which type or configuration of blocked molecule is used (i.e., blocked nucleic acid molecule, blocked primer molecule, or blocked guide molecule). The reaction mixture that flows in a stream from the left of FIG. 6D contains all components for the reaction (here, including the sample) except for the different RNP1 types. Again, in an alternative embodiment, the RNP2s could be contained in the reservoirs with the RNP1s and be added to the droplets along with the RNP1s.

In this sequential format embodiment, the different RNP1 types are kept separate before and after introduction into the reaction mixture flow. In this embodiment, different RNP1 types are introduced sequentially into a channel in a “burst” mode, with each different RNP1 type being introduced in a batch form controlled by, e.g., valves connected to reservoirs of RNP1s. Here, the reservoirs and valves are arranged linearly on, e.g., a substrate that is easily coupled to and removed from the microfluidic droplet system. The reservoirs and valves are all connected to a secondary channel which is then connected to the RNP1 introduction channel. In this embodiment, a first reservoir is opened where RNP1-A flows into the secondary channel, where this secondary channel then flows into the RNP1 introduction channel. The RNP1 introduction channel then meets and flows into the main flow channel through which the reaction mixture is flowing. As with FIGS. 6A, 6B, and 6C, after the RNP1-A liquid meets and combines in the main flow channel with the reaction mixture, oil (the “carrier fluid”) is then introduced from perpendicular oil introduction channels (here, above and below the main flow channel) in a flow focusing configuration, which pinches off aqueous droplets into the flow of the oil. Each aqueous droplet will comprise reaction mixture and some volume of the RNP1-A liquid burst or packet.

Again, droplets are generated that form a, e.g., picoliter-volume reaction partition (i.e., “mini-reactor”) for the RNP1-A in the signal boost cascade assay. In this embodiment, like that described in relation to FIG. 6C, it is not necessary for the droplets generated to comprise a Poisson or super Poisson distribution of RNP1s, as it is preferred that a number of RNP1s (here, RNP1-As) are present in each droplet and that there are several to many droplets generated for each RNP1 type, aiding in the statistical likelihood of positively identifying a target nucleic acid of interest present in the sample. The number of RNP1s present in each droplet and the number of droplets present for, e.g., RNP1-A, can be controlled by the concentration and volume of the RNP1-A burst and the pressure of the reaction mixture and RNP1-A burst flow. In this way, an entire reservoir of the RNP1-A burst can be used before moving on to a second reservoir with, e.g., an RNP1-B burst; alternatively, a portion of the RNP1-A burst in the first reservoir may be used initially, separated by slugs, then by, e.g., RNP1-B and so on through RNP1-N, all separated by slugs, then the cycle may be repeated using RNP1-A through RNP1-N for, e.g., identifying target nucleic acids of interest with greater statistical accuracy.

Here, as in the method described in relation to FIG. 6C, the different RNP1 type bursts are preferably separated by a “slug” or volume of separation fluid that does not comprise an RNP1 to clear any remnants of RNP1-A from the RNP1 introduction channel and to space out the different RNP1 types (RNP1-A, RNP1-B, RNP1-C, . . . RNP1-N) in the main flow channel and as the droplets are detected by the detector. As described above, the slug or separation fluid can be an aqueous fluid or carrier fluid. To minimize the amount of reaction mixture used, valve timing can be used to both stop the introduction of reaction mixture and start the introduction of separation fluid, which may be a carrier fluid. In another embodiment, the separation fluid may be an aqueous fluid comprising a colored or other compound that can be detected and the separation identified by the detector.

As with the embodiments described above, the aqueous droplets with RNP1s (and without RNP1s, i.e., the slugs) proceed through the main flow channel. If an RNP1 is activated by a target nucleic acid of interest binding to the gRNA of the RNP1, both cis-cleavage of the target nucleic acid of interest occurs and indiscriminate trans-cleavage activity of the nucleic acid-guided nuclease component of the RNP1 is activated, thereby cleaving other nucleic acids (specifically blocked molecules) in the reaction mixture. Here, a target nucleic acid of interest is detected by RNP1-C and fluorescence is present (denoted by stars). As the droplets comprising the RNP1-A pass the detector, no fluorescence is detected. Additionally, as the droplets comprising the slug or separation fluid pass the detector, no fluorescence is detected. Also, as the droplets comprising the RNP1-B pass the detector, no fluorescence is detected. However, as the droplets comprising the activated RNP1-C (and hence activated reporter) pass the detector, fluorescence is detected and RNP-C is identified as being activated; thus, the target nucleic acid of interest for RNP1-C is identified.

As described above, the detector can be any device or method appropriate for the reporter moiety used for interrogating a droplet as it passes through a detection region. In the present signal boost cascade assays using, e.g., a fluorescent reporter, one detector often used is an optical detector, such as a microscope or spectrophotometer and measured by a photo multiplier tube or other image processing or enhancement device, which may be coupled to a computer to digitize the signal may control the flow of the droplets via, e.g., valve action or an electroosmotic potential. FIG. 6D shows a simplified readout of fluorescence, with the fluorescence level of each drop (i.e., 3 RNP1-As, slugs, 3RNP1-Bs, slugs, and 2RNP1-Cs) detected.

FIG. 6E is a representation of a fifth exemplary microfluidic droplet system and method for performing the signal boost cascade assay in a massively multiplexed bulk format using RNP1/nucleic acid-encoded carrier beads which is particularly useful if the amount of sample is limited. As with the bulk format embodiments shown in FIGS. 6A and 6B and described above, the microfluidic droplet system carries out RNP1/carrier bead distribution into, e.g., picoliter-sized reagent droplets. Starting at left, an aqueous fluid comprising a reaction mixture (including RNP2s) flows through the main flow channel; however, unlike the preceding four exemplary embodiments, the sample is not added as a component of the reaction mixture. As with the embodiments described above and in relation to FIGS. 6A-6D, the reaction mixture will vary depending on which of the three embodiments of the signal boost cascade assay is employed; that is, the reaction mixture will vary by which type or configuration of blocked molecule is used (i.e., blocked nucleic acid molecule, blocked primer molecule, or blocked guide molecule). The reaction mixture that flows in a stream in the main flow channel from the left of FIG. 6E contains all components for the reaction here, excluding the sample and except for the library of carrier beads loaded with RNP1s.

To form the library of RNP1/carrier beads, RNP1s are formed in partitions, where different RNP1s targeting different target nucleic acids of interest (i.e., the different “RNP1 types”) are disposed into different partitions. As described above, in most embodiments the difference between RNP1 types is that different RNP1 types comprise different gRNA1s, where the nucleic acid-guided nuclease typically is the same for all RNP1 types (unless both RNA and DNA target nucleic acids are being detected in the same signal boost cascade assay). The different RNP1 types in the separate partitions are then coupled to nucleic acid-encoded carrier beads in the partitions, where each carrier bead is 1) optionally first linked to a photolabile (or other cleavable) linker; 2) then linked to a unique nucleic acid sequence which serves as a “compound code” for the RNP1 type, where the compound code for each different RNP1 type is different; and 3) finally linked to the RNP1s. Note that, as with the embodiment illustrated and described with respect to FIG. 6B, RNP2s could be included on the carrier beads.

After the carrier beads are loaded with a member of the library of RNP1 types, the RNP1/carrier beads (i.e., the library of RNP1/carrier beads) are pooled, thus mixing the different RNP1 type carrier beads. The library of RNP1/carrier beads are then introduced into the aqueous flow of the reaction mixture via the RNP1 introduction channel; here, preferably with a Poisson or super Poisson distribution of RNP1/carrier beads in the droplets. Moving right in FIG. 6E, oil (the “carrier fluid”) is then introduced from perpendicular oil introduction channels (here, above and below the main flow channel) in a flow focusing configuration, which pinches off aqueous droplets comprising the RNP1/carrier beads in the reaction mixture into the flow of the oil. Each aqueous droplet will comprise reaction mixture, but, if the flow of the RNP1/carrier bead library and the flow of the aqueous fluid comprising the reaction mixture is controlled, only approximately half to approximately 90% of the aqueous droplets will comprise an RNP1/carrier bead. By using this microfluidic approach, droplets are generated that form a reaction partition (i.e., the mini-reactor) for each of the RNP1/carrier beads in the signal boost cascade assay.

As with the embodiments described in relation to FIGS. 6A and 6B, the aqueous droplets with RNP1-loaded beads (and without RNP1-loaded beads) proceed through the flow channel and optionally are subjected to photochemical cleavage (via exposure to UV light) of the coded RNP1s from the carrier bead.

In this embodiment, after the addition of the oil carrier fluid to the main flow channel, an aqueous sample is delivered to the main flow channel and added to the carrier fluid at a pre-determined, desired rate so there is approximately one sample droplet formed per RNP1/carrier bead droplet. Downstream, the diameter of the main flow channel is reduced such that droplets—RNP1 droplets and sample droplets—are slowed down in the stream of carrier fluid and become in juxtaposition with or adjacent to one another. At this point, the droplets are merged or coalesced so that the droplets comprising the sample and the RNP1s merge and the RNP1s and the target nucleic acids of interest in the sample can react with one another. Droplet merger may be achieved using one of several methods, including 1) providing a localized electric field to coalesce the droplet, 2) providing locally a chemical that disrupts or destabilizes the surfactant in the continuous phase, such as, e.g., perfluoro-octanol, use of a textured surface in the flow path of the microfluidic channel as described in US Pub. No. 2019/0176152, or 3) constricting the diameter of the main flow channel to being the droplets into proximity, combined, e.g., with an electric field, surfactant-disrupting chemical or textured surface.

Continuing right in FIG. 6E, if RNP1 is activated by a target nucleic acid of interest from the sample binding to the gRNA, both cis-cleavage of the target nucleic acid of interest occurs and indiscriminate trans-cleavage activity of the nucleic acid-guided nuclease component of the RNP1 is activated thereby cleaving other nucleic acids (specifically blocked molecules) in the reaction mixture.

After the RNP1s react with target nucleic acids of interest from the sample, the now-merged aqueous droplets continue to flow through the microfluidic droplet system. Additional oil (not shown) may be optionally flowed into the existing oil carrier fluid flow in the main flow channel to regulate the space between droplets now containing completed signal boost cascade assay reactions and a detector is used to detect the level of fluorescence in each droplet. As described above, the detector can be any device or method for interrogating a droplet as it passes through a detection region. In the present signal boost cascade assays using, e.g., a fluorescent reporter, the detector is typically an optical detector, which may be coupled to a computer to digitize the signal may control the flow of the droplets via, e.g., valve action or an electroosmotic potential.

The droplets are detected, analyzed and sorted based on the intensity of a signal detected as the droplets pass through a detection region or window. Droplets having a level of reporter below a selected threshold or within a selected range are diverted into a predetermined outlet or reservoir. A droplet-sorting device may comprise a spectrophotometer where the fluorescent intensity of each droplet is read as it passes by the light beam, and the optical signal is collected and projected onto a cathode of a photomultiplier tube. Part of the light may be directed onto a charge-coupled device (CCD) camera for imaging. As the droplets pass by the spectrophotometer detection window, the droplets are directed to conduits that lead to the reservoirs that collect fluorescing droplets and empty droplets depending on, e.g., acoustic, electric, magnetic, and optical fields to impose forces to displace droplets for sorting.

Following sorting of the fluorescent-positive droplets into, e.g., a microtube or other container, the emulsion (aqueous droplets in oil) is broken thereby pooling the RNP1s and the nucleic acid barcodes that bound target nucleic acids of interest that are present in the droplet. The identity of the RNP1 can be determined by sequencing the nucleic acid barcodes in the pooled reaction mixture (here, sequence ATTGCG), which in turn identifies the target nucleic acid(s) of interest present in the sample. See MacConnell, et al., ACS Comb. Sci., 19:181-92 (2017) for a description of functional screens using DNA-encoded compound beads in a microfluidic droplet system.

### Applications of the Cascade Assay

The present disclosure describes cascade assays for detecting a target nucleic acid of interest in a sample that provide instantaneous or nearly instantaneous results, allow for massive multiplexing and minimum workflow, and yet provide accurate results at low cost. Moreover, the various embodiments of the cascade assay are notable in that, with the exception of the gRNAs in RNP1, the cascade assay components stay the same no matter what target nucleic acid(s) of interest are being detected. As described above, the cascade assay can be massively multiplexed for detecting several to many target nucleic acids of interest simultaneously without amplification of the nucleic acids in the sample. For example, the assay may be designed to detect several to many different pathogens (e.g., testing for many different pathogens in one assay), or the assay may be designed to detect one to several to many different sequences from the same pathogen (e.g., to increase specificity and sensitivity), or a combination of the two.

As described above, early and accurate identification of, e.g., infectious agents, microbe contamination, and variant nucleic acid sequences that indicate the presence of such diseases such as cancer or contamination by heterologous sources is important in order to select correct therapeutic treatment, identify tainted food, pharmaceuticals, cosmetics and other commercial goods; and to monitor the environment. The cascade assays described herein can be applied in diagnostics for, e.g., infectious disease (including but not limited to Covid, HIV, flu, the common cold, Lyme disease, STDs, chicken pox, diptheria, mononucleosis, hepatitis, UTIs, pneumonia, tetanus, rabies, malaria, dengue fever, Ebola, plague), for rapid liquid biopsies and companion diagnostics (biomarkers for cancers, early detection, progression, monitoring), prenatal testing (including but not limited to chromosomal abnormalities and genetic diseases such as sickle cell, including over-the-counter versions of prenatal testing assays), rare disease testing (achondroplasia, Addison's disease, al-antitrypsin deficiency, multiple sclerosis, muscular dystrophy, cystic fibrosis, blood factor deficiencies), SNP detection/DNA profiling/epigenetics, genotyping, low abundance transcript detection, labeling for cell or droplet sorting, in situ nucleic acid detection, sample prep, library quantification of NGS, screening biologics (including engineered therapeutic cells for genetic integrity and/or contamination), development of agricultural products, food compliance testing and quality control (e.g., detection of genetically modified products, confirmation of source for high value commodities, contamination detection), infectious disease in livestock, infectious disease in cash crops, livestock breeding, drug screening, personal genome testing including clinical trial stratification, personalized medicine, nutrigenomics, drug development and drug therapy efficacy, transplant compatibility and monitoring, environmental testing and forensics, and bioterrorism agent monitoring.

Target nucleic acids of interest are derived from samples as described in more detail above. Suitable samples for testing include, but are not limited to, any environmental sample, such as air, water, soil, surface, food, clinical sites and products, industrial sites and products, pharmaceuticals, medical devices, nutraceuticals, cosmetics, personal care products, agricultural equipment and sites, and commercial samples, and any biological sample obtained from an organism or a part thereof, such as a plant, animal, or microbe. In some embodiments, the biological sample is obtained from an animal subject, such as a human subject. A biological sample may be any solid or fluid sample obtained from, excreted by or secreted by any living organism, including, without limitation, single celled organisms, such as bacteria, yeast, protozoans, and amoebas among others, multicellular organisms including plants or animals, including samples from a healthy or apparently healthy human subject or a human patient affected by a condition or disease to be diagnosed or investigated, such as an infection with a pathogenic microorganism, such as a pathogenic bacteria or virus.

For example, a biological sample can be a biological fluid obtained from a human or non-human (e.g., livestock, pets, wildlife) animal, and may include but is not limited to blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface (e.g., a nasal or buccal swab).

The sample can be a viral or bacterial sample or a biological sample that has been minimally processed as described herein, e.g., only treated with a brief lysis step prior to detection. In other embodiments, minimal processing can include thermal lysis at an elevated temperature. In some embodiments, minimal processing can include treating the sample with chaotropic salts such as guanidine isothiocyanate or guanidine HCl and in some embodiments, minimal processing may include contacting the sample with reducing agents such as DTT or TCEP and EDTA to inactivate inhibitors and/or other nucleases present in the samples. In other embodiments, minimal processing for biofluids may include centrifuging the samples to obtain cell-debris free supernatant before applying the reagents.

The components of the cascade assay may be provided in various kits for testing at, e.g., point of care facilities, pandemic testing sites, and the like as long as a compatible microfluidic droplet system is available. In one aspect, the kit for detecting target nucleic acids of interest in a sample includes: several to many to massively multiplexed first ribonucleoprotein complexes (RNP1s) (separated into partitions), second ribonucleoprotein complexes (RNP2s), blocked nucleic acid molecules, and reporter moieties. The first complexes (RNP1s) comprise a first nucleic acid-guided nuclease and the first gRNAs (gRNA1 s) (again, separated into partitions), where the first gRNAs include the sequence complementary to the target nucleic acids of interest.

In a second aspect, the kit for detecting target nucleic acids of interest in sample includes: several to many to massively multiplexed first ribonucleoprotein complexes (RNP1s) (separated into partitions), second ribonucleoprotein complexes (RNP2s), template molecules, blocked primer molecules, a polymerase, NTPs, and reporter moieties. The first ribonucleoprotein complexes (RNP1s) comprise a first nucleic acid-guided nuclease and first gRNAs (gRNA1s), where the first gRNAs include a sequence complementary to the target nucleic acids of interest and where binding of RNP1 to one or more of the target nucleic acids of interest activates trans-cleavage activity of the first nucleic acid-guided nuclease.

In a third aspect, the kit for detecting target nucleic acids of interest in sample includes: several to many to massively multiplexed first ribonucleoprotein complexes (RNP1s) (separated into partitions) where the first ribonucleoprotein complexes (RNP1s) comprise a first nucleic acid-guided nuclease and first gRNAs (gRNA1s), second nucleic acid nucleases, blocked guide molecules, RNP2 activator nucleic acids, and reporter moieties. Again, the first gRNAs include a sequence complementary to the target nucleic acids of interest and where binding of RNP1 to one or more of the target nucleic acids of interest activates trans-cleavage activity of the first nucleic acid-guided nuclease.

Any of the kits described herein may further include a sample collection device, e.g., a syringe, lancet, nasal swab, or buccal swab for collecting a biological sample from a subject, and/or a sample preparation reagent, e.g., a lysis reagent. The kits can be used with an instrument comprising a sample prep module and a detection module. Each component of the kit may be in a separate container or two or more components may be in the same container although the RNP1s will be partitioned. In addition, the kit may further include instructions for use and other information.

### EXAMPLES

The following examples are put forth so as to provide those of ordinary skill in the art with a complete disclosure and description of how to make and use the present invention and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Preparation of Nucleic Acids of Interest

Mechanical lysis: Nucleic acids of interest may be isolated by various methods depending on the cell type and source (e.g., tissue, blood, saliva, environmental sample, etc.). Mechanical lysis is a widely used cell lysis method and may be used to extract nucleic acids from bacterial, yeast, plant and mammalian cells. Cells are disrupted by agitating a cell suspension with “beads” at high speeds (beads for disrupting various types of cells can be sourced from, e.g., OPS Diagnostics (Lebanon NJ, US) and MP Biomedicals (Irvine, CA, USA)). Mechanical lysis via beads begins with harvesting cells in a tissue or liquid, where the cells are first centrifuged and pelleted. The supernatant is removed and replaced with a buffer containing detergents as well as lysozyme and protease. The cell suspension is mixed to promote breakdown of the proteins in the cells and the cell suspension then is combined with small beads (e.g., glass, steel, or ceramic beads) that are mixed (e.g., vortexed) with the cell suspension at high speeds. The beads collide with the cells, breaking open the cell membrane with shear forces. After “bead beating”, the cell suspension is centrifuged to pellet the cellular debris and beads, and the supernatant may be purified via a nucleic acid binding column (such as the MagMAX™ Viral/Pathogen Nucleic Acid Isolation Kit from ThermoFisher (Waltham, MA, USA) and others from Qiagen (Hilden, Germany), TakaraBio (San Jose, CA, USA), and Biocomma (Shenzen, China)) to collect the nucleic acids (see the discussion of solid phase extraction below).

Solid phase extraction (SPE): Another method for capturing nucleic acids is through solid phase extraction. SPE involves a liquid and stationary phase, which selectively separates the target analyte (here, nucleic acids) from the liquid in which the cells are suspended based on specific hydrophobic, polar, and/or ionic properties of the target analyte in the liquid and the stationary solid matrix. Silica binding columns and their derivatives are the most commonly used SPE techniques, having a high binding affinity for DNA under alkaline conditions and increased salt concentration; thus, a highly alkaline and concentrated salt buffer is used. The nucleic acid sample is centrifuged through a column with a highly porous and high surface area silica matrix, where binding occurs via the affinity between negatively charged nucleic acids and positively charged silica material. The nucleic acids bind to the silica matrices, while the other cell components and chemicals pass through the matrix without binding. One or more wash steps typically are performed after the initial sample binding (i.e., the nucleic acids to the matrix), to further purify the bound nucleic acids, removing excess chemicals and cellular components non-specifically bound to the silica matrix. Alternative versions of SPE include reverse SPE and ion exchange SPE, and use of glass particles, cellulose matrices, and magnetic beads.

Thermal lysis: Thermal lysis involves heating a sample of mammalian cells, virions, or bacterial cells at high temperatures thereby damaging the cellular membranes by denaturizing the membrane proteins. Denaturizing the membrane proteins results in the release of intracellular DNA. Cells are generally heated above 90° C., however time and temperature may vary depending on sample volume and sample type. Once lysed, typically one or more downstream methods, such as use of nucleic acid binding columns for solid phase extraction as described above, are required to further purify the nucleic acids.

Physical lysis: Common physical lysis methods include sonication and osmotic shock. Sonication involves creating and then rupturing cavities or bubbles to release shockwaves, thereby disintegrating the cellular membranes of the cells. In the sonication process, cells are added into lysis buffer, often containing phenylmethylsulfonyl fluoride, to inhibit proteases. The cell samples are then placed in a water bath and a sonication wand is placed directly into the sample solution. Sonication typically occurs between 20-50 kHz, causing cavities to be formed throughout the solution as a result of the ultrasonic vibrations; subsequent reduction of pressure then causes the collapse of the cavity or bubble resulting in a large amount of mechanical energy being released in the form of a shockwave that propagates through the solution and disintegrates the cellular membrane. The duration of the sonication pulses and number of pulses performed varies depending on cell type and the downstream application. After sonication, the cell suspension typically is centrifuged to pellet the cellular debris and the supernatant containing the nucleic acids may be further purified by solid phase extraction as described above.

Another form of physical lysis is osmotic shock, which is most typically used with mammalian cells. Osmotic shock involves placing cells in DI/distilled water with no salt added. Because the salt concentration is lower in the solution than in the cells, water is forced into the cell causing the cell to burst, thereby rupturing the cellular membrane. The sample is typically purified and extracted by techniques such as e.g., solid phase extraction or other techniques known to those of skill in the art.

Chemical lysis: Chemical lysis involves rupturing cellular and nuclear membranes by disrupting the hydrophobic-hydrophilic interactions in the membrane bilayers via detergents. Salts and buffers (such as, e.g., Tris-HCl pH8) are used to stabilize pH during extraction, and chelating agents (such as ethylenediaminetetraacetic acid (EDTA)) and inhibitors (e.g., Proteinase K) are also added to preserve the integrity of the nucleic acids and protect against degradation. Often, chemical lysis is used with enzymatic disruption methods (see below) for lysing bacterial cell walls. In addition, detergents are used to lyse and break down cellular membranes by solubilizing the lipids and membrane proteins on the surface of cells. The contents of the cells include, in addition to the desired nucleic acids, inner cellular proteins and cellular debris. Enzymes and other inhibitors are added after lysis to inactivate nucleases that may degrade the nucleic acids. Proteinase K is commonly added after lysis, destroying DNase and RNase enzymes capable of degrading the nucleic acids. After treatment with enzymes, the sample is centrifuged, pelleting cellular debris, while the nucleic acids remain in the solution. The nucleic acids may be further purified as described above.

Another form of chemical lysis is the widely used procedure of phenol-chloroform extraction. Phenol-chloroform extraction involves the ability for nucleic acids to remain soluble in an aqueous solution in an acidic environment, while the proteins and cellular debris can be pelleted down via centrifugation. Phenol and chloroform ensure a clear separation of the aqueous and organic (debris) phases. For DNA, a pH of 7-8 is used, and for RNA, a more acidic pH of 4.5 is used.

Enzymatic lysis: Enzymatic disruption methods are commonly combined with other lysis methods such as those described above to disrupt cellular walls (bacteria and plants) and membranes. Enzymes such as lysozyme, lysostaphin, zymolase, and protease are often used in combination with other techniques such as physical and chemical lysis. For example, one can use cellulase to disrupt plant cell walls, lysosomes to disrupt bacterial cell walls and zymolase to disrupt yeast cell walls. Table 1 below lists exemplary commercial sample processing kits.

TABLE 1
Exemplary Commercial Sample and Nucleic Acid Processing Kits

| Manufacturer | Kit | Sample Type | Output | Lysing and extraction methods |
|---|---|---|---|---|
| Qiagen ® | DNeasy ™ Blood | small volumes | genomic | Isolation of Genomic DNA from Small |
| | & Tissue Kits | of blood | DNA | Volumes of Blood |
| | | dried blood | | 1. Uses Chemical and |
| | | spots | | Biological/Enzymatic lysis methods |
| | | urine | | 2. Uses SPE with Column Purification |
| | | tissues | | Isolation of Genomic DNA from Tissues |
| | | laser- | | 1. Uses Chemical and |
| | | microdissected | | Biological/Enzymatic lysis methods |
| | | tissues | | 2. Used to dissolve and lyse tissue sections |
| | | | | completely, higher temperature and |
| | | | | longer time incubations up to 24 hours are |
| | | | | used |
| Qiagen ® | QIAamp ® UCP | whole blood | microbial | Specific pretreatment protocols are |
| | Pathogen | swabs | DNA | suggested depending on sample type with |
| | Mini Handbook | cultures -- | | or without the use of kits for Mechanical |
| | microbial DNA | pelleted | | Lysis Method before downstream |
| | purification | microbial cells | | applications. |
| | | body fluids | | Downstream applications contain: |
| | | | | 1. Chemical and Biological/Enzymatic |
| | | | | lysis methods |
| | | | | 2. SPE with Column Purification |
| Qiagen ® | QIAamp ® Viral | plasma and | viral DNA | 1. Uses Chemical lysis methods |
| | RNA Kits | serum | | 2. Uses SPE with Column Purification |
| | | CSF | | |
| | | urine | | |
| | | other cell-free | | |
| | | body fluids | | |
| | | cell-culture | | |
| | | supernatants | | |
| | | swabs | | |
| Zymo | Quick- | whole blood | genomic | 1. Uses chemical lysis methods |
| Research ™ | DNA ™Microprep | plasma | DNA | 2. Uses SPE with column purification |
| | Kit | serum | | |
| | | body fluids | | |
| | | buffy coat | | |
| | | lymphocytes | | |
| | | swabs | | |
| | | cultured cells | | |
| Zymo | Quick-DNA ™ | A. fumigatus | Microbial | Uses Bead lysis and pretreatment with: |
| Research ™ | Fungal/Bacterial | C. albicans | DNA | 1. Chemical lysis methods with |
| | Miniprep Kit | N. crassa | | chaotropic salts |
| | | S. cerevisiae | | 2. NAE with SPE with silica matrices |
| | | S. pombe | | |
| | | mycelium | | |
| | | Gram positive | | |
| | | bacteria | | |
| | | Gram negative | | |
| | | bacteria | | |

### Example II: RNP Formation

For RNP complex formation, 250 nM of LbCas12a nuclease protein was incubated with 375 nM of a target specific gRNA in 1× Buffer (10 mM Tris-HCl, 100 pg/mL BSA) with 2-15 mM MgCl₂ at 25° C. for 20 minutes. The total reaction volume was 2 μL. Other ratios of LbCas12a nuclease to gRNAs were tested, including 1:1, 1:2 and 1:5. The incubation temperature ranged from 16° C.-37° C., and the incubation time ranged from 10 minutes to 4 hours.

### Example III: Blocked Nucleic Acid Molecule Formation

Ramp cooling: For formation of the secondary structure of blocked nucleic acid molecules, 2.5 μM of a blocked nucleic acid molecule was combined with a T50 buffer (20 mM Tris HCl, 50 mM NaCl) with 10 mM MgCl₂ for a total volume of 50 μL. The reaction was heated to 95° C. at 1.6° C./second and incubated at 95° C. for 5 minutes to dehybridize any secondary structures. Thereafter, the reaction was cooled to 37° C. at 0.015° C./second to form the desired secondary structure.

Snap cooling: For formation of the secondary structure of blocked nucleic acid molecules, 2.5 μM of a blocked nucleic acid molecule was combined with a T50 buffer (20 mM Tris HCl, 50 mM NaCl) with 10 mM MgCl₂ for a total volume of 50 μL. The reaction was heated to 95° C. at 1.6° C./second and incubated at 95° C. for 5 minutes to dehybridize any secondary structures. Thereafter, the reaction was cooled to room temperature by removing the heat source to form the desired secondary structure.

Snap cooling on ice: For formation of the secondary structure of blocked nucleic acid molecules, 2.5 μM of a blocked nucleic acid molecule was combined with a T50 buffer (20 mM Tris HCl, 50 mM NaCl) with 10 mM MgCl₂ for a total volume of 50 μL. The reaction was heated to 95° C. at 1.6° C./second and incubated at 95° C. for 5 minutes to dehybridize any secondary structures. Thereafter, the reaction was cooled to room temperature by placing the reaction tube on ice to form the desired secondary structure.

### Example IV: Reporter Moiety Formation

The reporter moieties used in the reactions herein were single-stranded DNA oligonucleotides 5-9 bases in length (e.g., with sequences of TTATT, TTTATTT, ATTAT, ATTTATTTA, AAAAA, or AAAAAAAAA) with a fluorophore and a quencher attached on the 5′ and 3′ ends, respectively. In one example using a Cas12a cascade, the fluorophore was FAM-6 and the quencher was IOWA BLACK® (Integrated DNA Technologies, Coralville, IA). In another example using a Cas13 cascade, the reporter moieties were single-stranded RNA oligonucleotides 5-10 bases in length (e.g., r(U)n, r(UUAUU)n, r(A)n).

### Example V: Cascade Assay

Format I (final reaction mixture components added at the same time): RNP1 was assembled using the LbCas12a nuclease and a gRNA for the Methicillin resistant Staphylococcus aureus (MRSA) DNA according to the RNP complex formation protocol described in Example II (for this sequence, see Example VI). Briefly, 250 nM LbCas12a nuclease was assembled with 375 nM of the MRSA-target specific gRNA. Next, RNP2 was formed using the LbCas12a nuclease and a gRNA specific for a selected blocked nucleic acid molecule using 500 nM LbCas12a nuclease assembled with 750 nM of the blocked nucleic acid-specific gRNA incubated in 1×NEB 2.1 Buffer (New England Biolabs, Ipswich, MA) with 5 mM MgCl₂ at 25° C. for 20-40 minutes. Following incubation, RNP1s were diluted to a concentration of 75 nM LbCas12a: 112.5 nM gRNA. Thereafter, the final reaction was carried out in 1× Buffer, with 500 nM of the ssDNA reporter moiety, 1×ROX dye (Thermo Fisher Scientific, Waltham, MA) for passive reference, 2.5 mM MgCl₂, 4 mM NaCl, 15 nM LbCas12a: 22.5 nM gRNA RNP1, 20 nM LbCas12a: 35 nM gRNA RNP2, and 50 nM blocked nucleic acid molecule in a total volume of 9 μL. 1 μL of MRSA DNA target (with samples having as low as three copies and as many as 30000 copies—see FIGS. 6-14) was added to make a final volume of 10 μL. The final reaction was incubated in a thermocycler at 25° C. with fluorescence measurements taken every 1 minute.

Format II (RNP1 and MRSA target pre-incubated before addition to final reaction mixture): RNP1 was assembled using the LbCas12a nuclease and a gRNA for the MRSA DNA according to the RNP formation protocol described in Example II (for this sequence, see Example VI). Briefly, 250 nM LbCas12a nuclease was assembled with 375 nM of the MRSA-target specific gRNA. Next, RNP2 was formed using the LbCas12a nuclease and a gRNA specific for a selected blocked nucleic acid molecule using 500 nM LbCas12a nuclease assembled with 750 nM of the blocked nucleic acid-specific gRNA incubated in 1×NEB 2.1 Buffer (New England Biolabs, Ipswich, MA) with 5 mM MgCl₂ at 25° C. for 20-40 minutes. Following incubation, RNP1s were diluted to a concentration of 75 nM LbCas12a: 112.5 nM gRNA. After dilution, the formed RNP1 was combined with 1 μL of MRSA DNA target and incubated at 16° C.-37° C. for up to 10 minutes to activate RNP1. The final reaction was carried out in 1× Buffer, with 500 nM of the ssDNA reporter moiety, 1×ROX dye (Thermo Fisher Scientific, Waltham, MA) for passive reference, 2.5 mM MgCl₂, 4 mM NaCl, the pre-incubated and activated RNP1, 20 nM LbCas12a: 35 nM gRNA RNP2, and 50 nM blocked nucleic acid molecule in a total volume of 9 μL. The final reaction was incubated in a thermocycler at 25° C. with fluorescence measurements taken every 1 minute.

Format III (RNP1 and MRSA target pre-incubated before addition to final reaction mixture and blocked nucleic acid molecule added to final reaction mixture last): RNP1 was assembled using the LbCas12a nuclease and a gRNA for the MRSA DNA according to the RNP complex formation protocol described in Example II (for this sequence, see Example VI). Briefly, 250 nM LbCas12a nuclease was assembled with 375 nM of the MRSA-target specific gRNA. Next, RNP2 was formed using the LbCas12a nuclease and a gRNA specific for a selected blocked nucleic acid molecule using 500 nM LbCas12a nuclease assembled with 750 nM of the blocked nucleic acid-specific gRNA incubated in 1×NEB 2.1 Buffer (New England Biolabs, Ipswich, MA) with 5 mM MgCl₂ at 25° C. for 20-40 minutes. Following incubation, RNP1s were diluted to a concentration of 75 nM LbCas12a: 112.5 nM gRNA. After dilution, the formed RNP1 was combined with 1 μL of MRSA DNA target and incubated at 16° C.-37° C. for up to 10 minutes to activate RNP1. The final reaction was carried out in 1× Buffer, with 500 nM of the ssDNA reporter moiety, 1×ROX dye (Thermo Fisher Scientific, Waltham, MA) for passive reference, 2.5 mM MgCl₂, 4 mM NaCl, the pre-incubated and activated RNP1, and 20 nM LbCas12a: 35 nM gRNA RNP2 in a total volume of 9 μL. Once the reaction mixture was made, 1 μL (50 nM) blocked nucleic acid molecule was added for a total volume of 10 μL. The final reaction was incubated in a thermocycler at 25° C. with fluorescence measurements taken every 1 minute.

### Example VI: Detection of MRSA and Test Reaction Conditions

To detect the presence of Methicillin resistant Staphylococcus aureus (MRSA) and determine the sensitivity of detection with the cascade assay, titration experiments with a MRSA DNA target nucleic acid of interest were performed. The MRSA DNA sequence (NCBI Reference Sequence NC: 007793.1, is as follows.

SEQ ID NO: 6:
ATGAAAAAGATAAAAATTGTTCCACTTATTTTAATAGTTGTAGTTGTCG
GGTTTGGTATATATTTTTATG
CTTCAAAAGATAAAGAAATTAATAATACTATTGATGCAATTGAAGATAA
AAATTTCAAACAAGTTTATAA
AGATAGCAGTTATATTTCTAAAAGCGATAATGGTGAAGTAGAAATGACT
GAACGTCCGATAAAAATATAT
AATAGTTTAGGCGTTAAAGATATAAACATTCAGGATCGTAAAATAAAAA
AAGTATCTAAAAATAAAAAAC
GAGTAGATGCTCAATATAAAATTAAAACAAACTACGGTAACATTGATCG
CAACGTTCAATTTAATTTTGT
TAAAGAAGATGGTATGTGGAAGTTAGATTGGGATCATAGCGTCATTATT
CCAGGAATGCAGAAAGACCAA
AGCATACATATTGAAAATTTAAAATCAGAACGTGGTAAAATTTTAGACC
GAAACAATGTGGAATTGGCCA
ATACAGGAACAGCATATGAGATAGGCATCGTTCCAAAGAATGTATCTAA
AAAAGATTATAAAGCAATCGC
TAAAGAACTAAGTATTTCTGAAGACTATATCAAACAACAAATGGATCAA
AATTGGGTACAAGATGATACC
TTCGTTCCACTTAAAACCGTTAAAAAAATGGATGAATATTTAAGTGATT
TCGCAAAAAAATTTCATCTTA
CAACTAATGAAACAGAAAGTCGTAACTATCCTCTAGGAAAAGCGACTTC
ACATCTATTAGGTTATGTTGG
TCCCATTAACTCTGAAGAATTAAAACAAAAAGAATATAAAGGCTATAAA
GATGATGCAGTTATTGGTAAA
AAGGGACTCGAAAAACTTTACGATAAAAAGCTCCAACATGAAGATGGCT
ATCGTGTCACAATCGTTGACG
ATAATAGCAATACAATCGCACATACATTAATAGAGAAAAAGAAAAAAGA
TGGCAAAGATATTCAACTAAC
TATTGATGCTAAAGTTCAAAAGAGTATTTATAACAACATGAAAAATGAT
TATGGCTCAGGTACTGCTATC
CACCCTCAAACAGGTGAATTATTAGCACTTGTAAGCACACCTTCATATG
ACGTCTATCCATTTATGTATG
GCATGAGTAACGAAGAATATAATAAATTAACCGAAGATAAAAAAGAACC
TCTGCTCAACAAGTTCCAGAT
TACAACTTCACCAGGTTCAACTCAAAAAATATTAACAGCAATGATTGGG
TTAAATAACAAAACATTAGAC
GATAAAACAAGTTATAAAATCGATGGTAAAGGTTGGCAAAAAGATAAAT
CTTGGGGTGGTTACAACGTTA
CAAGATATGAAGTGGTAAATGGTAATATCGACTTAAAACAAGCAATAGA
ATCATCAGATAACATTTTCTT
TGCTAGAGTAGCACTCGAATTAGGCAGTAAGAAATTTGAAAAAGGCATG
AAAAAACTAGGTGTTGGTGAA
GATATACCAAGTGATTATCCATTTTATAATGCTCAAATTTCAAACAAAA
ATTTAGATAATGAAATATTAT
TAGCTGATTCAGGTTACGGACAAGGTGAAATACTGATTAACCCAGTACA
GATCCTTTCAATCTATAGCGC
ATTAGAAAATAATGGCAATATTAACGCACCTCACTTATTAAAAGACACG
AAAAACAAAGTTTGGAAGAAA
AATATTATTTCCAAAGAAAATATCAATCTATTAACTGATGGTATGCAAC
AAGTCGTAAATAAAACACATA
AAGAAGATATTTATAGATCTTATGCAAACTTAATTGGCAAATCCGGTAC
TGCAGAACTCAAAATGAAACA
AGGAGAAACTGGCAGACAAATTGGGTGGTTTATATCATATGATAAAGAT
AATCCAAACATGATGATGGCT
ATTAATGTTAAAGATGTACAAGATAAAGGAATGGCTAGCTACAATGCCA
AAATCTCAGGTAAAGTGTATG
ATGAGCTATATGAGAACGGTAATAAAAAATACGATATAGATGAATAA

Briefly, an RNP1 was preassembled with a gRNA sequence designed to target MRSA DNA. Specifically, RNP1 was designed to target a 20 bp region of the mecA gene of MRSA: TGTATGGCATGAGTAACGAA (SEQ ID NO: 7). An RNP2 was preassembled with a gRNA sequence designed to target the unblocked nucleic acid molecule that results from unblocking (i.e., linearizing) blocked nucleic acid molecule. The reaction mix contained the preassembled RNP1, preassembled RNP2, and a blocked nucleic acid molecule, in a buffer (pH of about 8) containing 4 mM MgCl₂ and 101 mM NaCl.

The blocked nucleic acid molecule used herein had a secondary structure free energy value of −5.84 kcal/mol and relatively short self-hybridizing, double-stranded regions of 5 bases and 6 bases. Results were achieved for detection of 3E4 copies, 30 copies, 3 copies and 0 copies of the mecA gene of MRSA (n=3) at 25° C. with varying concentrations of blocked nucleic acid, RNP2 and reporter moiety. When 100 nM blocked nucleic acid molecules, 10 nM RNP2s and 500 nM reporter moieties are used, the ratio of blocked nucleic acid molecules to RNP2s was 10:1. With 3E4 copies, nearly 100% of the reporters were cleaved at t=0 with a signal-to-noise ratio of 28.06 at 0 minutes, a signal-to-noise ratio of 24.23 at 5 minutes, and a signal-to-noise ratio of 21.01 at 10 minutes (data not shown). Additionally, the signal-to-noise ratios for detection with 30 copies of MRSA target was 12.45 at 0 minutes, 14.07 at 5 minutes and 16.16 at 10 minutes; and the signal-to-noise ratios for detection with 3 copies of MRSA target is 1.79 at 0 minutes, 1.64 at 5 minutes and is 2.04 at 10 minutes. Note the measured fluorescence at 0 copies increases only slightly over the 10- and 30-minutes intervals, resulting in a flat negative. A flat negative (the results obtained over the time period for 0 copies) demonstrated that there is very little non-specific or undesired signal generation in the system.

While certain embodiments have been described, these embodiments have been presented by way of example only and are not intended to limit the scope of the present disclosures. Indeed, the novel methods, apparatuses, modules, instruments and systems described herein can be embodied in a variety of other forms; furthermore, various omissions, substitutions and changes in the form of the methods, apparatuses, modules, instruments and systems described herein can be made without departing from the spirit of the present disclosures. The accompanying claims and their equivalents are intended to cover such forms or modifications as would fall within the scope and spirit of the present disclosures.

We claim:

1. A method for identifying one or more target nucleic acids of in a sample comprising the steps of:
designing first guide nucleic acids (gRNA1s) complementary to the target nucleic acids of interest;
forming first ribonucleoprotein complexes (RNP1s) comprising a first nucleic acid-guided nuclease and the gRNA1s; wherein the first nucleic acid-guided nuclease exhibits both cis- and trans-cleavage activity and wherein the RNP1s are formed in partitions where different partitions comprise different gRNA1 sequences;
providing a reaction mixture comprising:
the sample;
second ribonucleoprotein complexes (RNP2s) comprising a second nucleic acid-guided nuclease and a second gRNA that is not complementary to the target nucleic acids of interest; wherein the second nucleic acid-guided nuclease exhibits both cis- and trans-cleavage activity;
a plurality of blocked nucleic acid molecules comprising a sequence complementary to the second gRNA, wherein the blocked nucleic acid molecules comprise: a first region recognized by the second gRNA of the RNP2 complex and one or more second regions not complementary to the first region forming at least one loop; and
a plurality of reporter moieties comprising a detectable signal wherein the detectable signal is activated by the trans-cleavage activity of the RNP1s and/or RNP2s;
providing a microfluidic droplet system comprising a main flow channel, an RNP1 introduction channel and at least one oil flow channel;
introducing a first aqueous fluid through the main flow channel, wherein the first aqueous fluid comprises the reaction mixture;
introducing a second aqueous fluid through the RNP1 introduction channel into the first aqueous fluid in the main flow channel, wherein the second aqueous fluid comprises RNP1 complexes with a first gRNA;
following introduction of the second aqueous fluid into the main flow channel, introducing a carrier fluid through one or more carrier fluid introduction channels into the main flow channel thereby forming aqueous droplets comprising reaction mixture and RNP1s in the carrier fluid;
providing conditions for the one or more target nucleic acids of interest in the sample, if present, to bind to the RNP1s; and
detecting the detectable signal, if present, in the aqueous droplets.

2. The method of claim 1, further comprising the steps of sorting the droplets with detectable signal from the aqueous droplets without detectable signal; pooling the droplets with detectable signal; separating the droplets with detectable signal from carrier fluid; and sequencing the nucleic acid barcodes present in the droplets with detectable signal.

3. The method of claim 1, further comprising, after the step of flowing the second aqueous fluid through the RNP1 introduction channel, the step of flowing a first slug fluid through the RNP1 introduction channel and into the first aqueous fluid in the main flow channel, wherein the first slug fluid does not comprise RNP1s.

4. The method of claim 3, wherein the first slug fluid is aqueous.

5. The method of claim 4, wherein the first slug fluid has a detectable property.

6. The method of claim 3, wherein the first slug fluid is carrier fluid.

7. The method of claim 3, further comprising, after flowing the first slug fluid through the RNP1 introduction channel, the step of flowing a third aqueous fluid through the RNP1 flow channel and into the first aqueous fluid in the main flow channel, wherein the third aqueous fluid comprises RNP1 complexes with a second gRNA.

8. The method of claim 7, further comprising, after the step of flowing the third aqueous fluid through the RNP1 introduction channel, the step of flowing a second slug fluid through the RNP1 introduction channel and into the first aqueous fluid in the main flow channel, wherein the second slug fluid does not comprise RNP1s.

9. The method of claim 8, further comprising, after flowing the second slug fluid through the RNP1 introduction channel, the step of flowing a fourth aqueous fluid through the RNP1 flow channel and into the first aqueous fluid in the main flow channel, wherein the fourth aqueous fluid comprises RNP1 complexes with a third gRNA.

10. The method of claim 1, wherein the forming step is performed where the partitions are reservoirs coupled by valves to the RNP1 introduction channel.

11. The method of claim 1, wherein the carrier fluid is a non-polar hydrophobic fluid.

12. The method of claim 11, wherein the non-polar hydrophobic fluid is a fluorinated oil.

13. The method of claim 1, wherein the aqueous droplets comprising reaction mixture and RNP1s in the carrier fluid have a volume of approximately 50 fL to 10 nL.

14. The method of claim 13, wherein the aqueous droplets comprising reaction mixture and RNP1s in the carrier fluid have a volume of approximately 1 pL to 1 nL.

15. The method of claim 14, wherein the aqueous droplets comprising reaction mixture and RNP1s in the carrier fluid have a volume of approximately 10 pL to 900 pL.

16. The method of claim 15, wherein the aqueous droplets comprising reaction mixture and RNP1s in the carrier fluid have a volume of approximately 100 pL to 500 pL.

17. The method of claim 1, wherein the aqueous droplets comprising reaction mixture and RNP1s in the carrier fluid have a volume of less than 1 nL.

18. The method of claim 17, wherein the aqueous droplets comprising reaction mixture and RNP1s in the carrier fluid have a volume of less than 500 pL.

19. The method of claim 1, wherein the aqueous droplets comprising reaction mixture and RNP1s in the carrier fluid flow through the main flow channel at a rate of approximately 10 droplets/minute to 100 droplets/minute.

20. The method of claim 1, wherein the detectable signal is a fluorescent signal.

21. The method of claim 1, wherein the microfluidic droplet system further comprises integral imaging and droplets with detectable signal may be sorted from droplets without detectable signal.

22. The method of claim 1, wherein the microfluidic droplet system comprises two carrier fluid introduction channels configured to provide flow focusing.

23. The method of claim 1, wherein there are five different RNP1s sequentially introduced into the RNP1 introduction channel.

24. The method of claim 23, wherein there are 10 different RNP1s sequentially introduced into the RNP1 introduction channel.

25. The method of claim 24, wherein there are 20 different RNP1s sequentially introduced into the RNP1 introduction channel.

26. The method of claim 25, wherein there are 100 different RNP1s sequentially introduced into the RNP1 introduction channel.

27. The method of claim 25, wherein there are 250 different RNP1s sequentially introduced into the RNP1 introduction channel.

28. The method of claim 1, wherein there are reservoirs coupled by valves to the RNP1 introduction channel.

29. The method of claim 28, wherein there are at least five reservoirs coupled by valves to the RNP1 introduction channel, wherein four reservoirs comprise different RNP1s and one reservoir comprises slug fluid.

30. The method of claim 29, wherein there are at least eleven reservoirs coupled by valves to the RNP1 introduction channel, wherein ten reservoirs comprise different RNP1s and one reservoir comprises slug fluid.


SEQUENCE LISTING (7 sequences)

SEQ ID NO: 1 - protein, 10 aa, unidentified
CGLVPAGSGP

SEQ ID NO: 2 - protein, 12 aa, synthetic construct
SLLKSRMVPNFN

SEQ ID NO: 3 - protein, 12 aa, synthetic construct
SLLIARRMPNFN

SEQ ID NO: 4 - protein, 12 aa, synthetic construct
SKLVQASASGVN

SEQ ID NO: 5 - protein, 12 aa, synthetic construct
SSYLKASDAPDN

SEQ ID NO: 6 - DNA, 2007 bp, Staphylococcus aureus
  source 1..2007: mol_type = genomic DNA
atgaaaaagataaaaattgttccacttattttaatagttgtagttgtcgggtttggtatatatttttatgcttcaaaagataaagaaattaataatactattgatgcaattgaagataaaaatttcaaacaagtttataaagatagcagttatatttctaaaagcgataatggtgaagtagaaatgactgaacgtccgataaaaatatataatagtttaggcgttaaagatataaacattcaggatcgtaaaataaaaaaagtatctaaaaataaaaaacgagtagatgctcaatataaaattaaaacaaactacggtaacattgatcgcaacgttcaatttaattttgttaaagaagatggtatgtggaagttagattgggatcatagcgtcattattccaggaatgcagaaagaccaaagcatacatattgaaaatttaaaatcagaacgtggtaaaattttagaccgaaacaatgtggaattggccaatacaggaacagcatatgagataggcatcgttccaaagaatgtatctaaaaaagattataaagcaatcgctaaagaactaagtatttctgaagactatatcaaacaacaaatggatcaaaattgggtacaagatgataccttcgttccacttaaaaccgttaaaaaaatggatgaatatttaagtgatttcgcaaaaaaatttcatcttacaactaatgaaacagaaagtcgtaactatcctctaggaaaagcgacttcacatctattaggttatgttggtcccattaactctgaagaattaaaacaaaaagaatataaaggctataaagatgatgcagttattggtaaaaagggactcgaaaaactttacgataaaaagctccaacatgaagatggctatcgtgtcacaatcgttgacgataatagcaatacaatcgcacatacattaatagagaaaaagaaaaaagatggcaaagatattcaactaactattgatgctaaagttcaaaagagtatttataacaacatgaaaaatgattatggctcaggtactgctatccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaaaatattatttccaaagaaaatatcaatctattaactgatggtatgcaacaagtcgtaaataaaacacataaagaagatatttatagatcttatgcaaacttaattggcaaatccggtactgcagaactcaaaatgaaacaaggagaaactggcagacaaattgggtggtttatatcatatgataaagataatccaaacatgatgatggctattaatgttaaagatgtacaagataaaggaatggctagctacaatgccaaaatctcaggtaaagtgtatgatgagctatatgagaacggtaataaaaaatacgatatagatgaataa

SEQ ID NO: 7 - RNA, 20 nt, synthetic construct
  source 1..20: mol_type = other RNA
tgtatggcatgagtaacgaa